(19) United States
(12) Patent Application Publication
(10) Pub. No.: US 2001/0032368 A1
(43) Pub. Date: Oct. 25, 2001

(54) Dye compositions comprising at least one nonionic compound and uses thereof

(76) Inventors: Eric Bone, Rueil Malmaison (FR); Harumi Mori, Tokyo (JP); Hidetoshi Yamada, Tokyo (JP)

Correspondence Address: Thomas L. Irving, FINNEGAN, HENDERSON, FARABOW, GARRETT & DUNNER, L.L.P., 1300 I Street, N.W., Washington, DC, 20005-3315, US

(21) Appl. No.: 09/727,585

(22) Filed: Dec. 4, 2000

(30) Foreign Application Priority Data
Dec. 3, 1999 (JP) HEI 11 345546

(51) Int. Cl. A61K 7/13

(52) U.S. Cl. 8/405; 8/406; 8/409; 8/410; 8/421

(57) ABSTRACT

Dye compositions comprising (1) at least one colorant chosen from oxidation dyes and direct dyes, wherein said oxidation dyes are chosen from bases, couplers, and bases and couplers, and (2) at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100. One use of such compositions is for the dyeing of at least one keratin fiber, such as human keratin fibers like hair. Processes and devices for dyeing using the aforesaid compositions.

The present invention relates to dye compositions comprising (1) at least one colorant chosen from oxidation dyes and direct dyes, wherein said oxidation dyes are chosen from bases, couplers, and bases and couplers, and (2) at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100. One use of such compositions is for the dyeing keratin fibers, such as human keratin fibers like hair.

Two types of coloration can be distinguished in the field of hair treatment, direct coloration and oxidation coloration.

The first type of coloration, direct coloration, otherwise known as semi-permanent or temporary coloration, involves dyes capable of giving the hair's natural color a more or less pronounced color change which may withstand shampooing up to several times. These colorants are known as direct dyes, and they can be used with or without an oxidizing agent. If a lightening direct coloration is desired, then the colorant can be used in the presence of an oxidizing agent. The lightening coloration is carried out by applying to the hair a mixture, prepared at the time of use, of at least one direct dye and at least one oxidizing agent. As a result, by, for example, lightening the melanin in the hair, it is possible to obtain at least one advantageous effect such as a unified color in the case of gray hair, and accentuated color in the case of naturally pigmented hair.

The second type of coloration, permanent coloration or oxidation coloration, involves dyes known as “oxidation” dyes comprising at least one agent chosen from oxidation dye precursors and oxidation dye couplers. Oxidation dye precursors, commonly known as “oxidation bases”, are initially colorless or weakly colored compounds which develop their dyeing power on the hair in the presence of oxidizing agents added at the time of use, leading to the formation of colored compounds and dyes. The formation of these colored compounds and dyes results either from an oxidative condensation of the “oxidation bases” with themselves or from an oxidative condensation of the “oxidation bases” with coloration modifier compounds commonly known as “couplers”, which are generally present in the dye compositions used in oxidation coloration.

The variety of compositions that can be employed in oxidation coloration, chosen from oxidation bases, oxidation couplers and mixtures of oxidation bases and couplers, contributes to a pallet possibly very rich in color.

To vary further the shades obtainable by the oxidation dyes, and to enrich the glints, direct dyes may be added thereto.

Traditional thickeners, which can provide a gelling effect when diluted by water and/or surfactants, have been used conventionally to localize the dye product as applied on hair, so that the dye product does not touch the face or the area outside the area to be dyed. Such thickeners, chosen as appropriate, for example include crosslinked polyacrylic acid, hydroxyethylcelluloses, waxes, and mixtures of nonionic surfactants of HLB (Hydrophilic Lipophilic Balance).

The inventors have discovered that the ingredients of the traditional thickener type, surfactant and solvent, can generally decrease the holding of the dye on the fibers. This could account for dyeing resulting in dull shades and for the need to employ a larger quantity of the dye, solvent and/or surfactant to be used to dissolve the dye, especially when stronger shades are needed.

Furthermore, the inventors have discovered instances wherein, after mixing with at least one oxidizing agent, dyeing compositions comprising at least one dye and at least one of the above-described ingredients tended to lose part of their gelling character and tended to exhibit undesirable fluidity.

The inventors have discovered that it is possible to overcome at least one of these drawbacks. By introducing an effective amount of a particular nonionic compound (1) to the dyeing compositions, (2) to the oxidizing composition (when it relates to oxidation dyeing or lightening direct dyeing), or (3) to the two compositions at the same time, it is possible to obtain coloring compositions that tend to achieve at least one of the following properties: (1) resist flow, (2) remain more localized at the application location, and (3) obtain stronger and more chromatic (luminous) shades.

One aspect of the present invention is to provide dyeing compositions comprising, in a medium suitable for dyeing, (1) at least one dye, and (2) at least one nonionic compound of the formula R—(OCH₂CH₂)ₙ—OR′, wherein R is chosen from C₁₀-C₃₀ alkyl groups, R′ is chosen from C₁₀-C₃₀ alkyl groups optionally substituted with a hydroxyl group, and n is an integer ranging from 1 to 100. One use of such compositions is for the dyeing keratin fibers, such as human keratin fibers like hair.

According to the present invention, it is possible to provide dyeing compositions having a suitably high viscosity by utilizing an effective amount, which may be relatively small, of at least one nonionic compound which improves the quality of use at the time of application of said dyeing compositions.

It is also possible to reduce the consumption of the surfactants.

The invention also makes it possible to reduce the quantity of the active colorants used in the dyeing compositions compared with the classical technique.

Furthermore, at least certain compositions according to the invention make it possible to obtain shades tending to have at least one of little selectivity and good resistance with respect to at least one agent chosen from chemical agents (shampoos, permanent agents) and natural agents (light, perspiration).

Another aspect of the present invention relates to a composition ready to use for dyeing keratin fibers, wherein said composition comprises:

at least one composition (A) comprising, in a medium appropriate for dyeing, at least one colorant chosen from oxidation dyes and direct dyes, wherein said oxidation dyes are chosen from bases, couplers, and bases and couplers, and

at least one composition (B) comprising at least one oxidizing agent, and

at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100, wherein said at least one nonionic compound is present in at least one of said at least one composition (A), in at least one of said at least one composition (B), and in at least one of said at least one composition (A) and in at least one of said at least one composition (B).

The present invention also provides a dyeing process of keratin fibers, such as human keratin fibers like hair, comprising applying to said keratin fibers at least one composition comprising, in a medium appropriate for dyeing, at least one direct dye and at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100, and optionally leaving said composition to act on the fibers for a time, such as a resting time, ranging from 3 to 60 minutes approximately, rinsing said keratin fibers, optionally washing said keratin fibers, rinsing said keratin fibers again after said optional washing, and drying said keratin fibers. Such a dyeing process can be performed on said at least one keratin fiber when said keratin fibers are dry or wet.

Another aspect of the present invention also relates to a process of dyeing keratin fibers, such as human keratin fibers like hair, comprising applying on said keratin fibers:

at least one composition (A) comprising, in a medium appropriate for dyeing, at least one colorant chosen from oxidation dyes and direct dyes, wherein said oxidation dyes are chosen from bases, couplers, and bases and couplers, and

at least one oxidizing composition (B), said dyeing being developed at alkaline, neutral or acid pH,

wherein said oxidizing composition (B) is mixed at the time of use, such as just at the moment of use, with the composition (A) or said oxidizing composition (B) is applied sequentially to composition (A) without intermediate rinsing, and

at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100, wherein said at least one nonionic compound is present in at least one of said at least one composition (A), in at least one of said at least one composition (B), and in at least one of said at least one composition (A) and in at least one of said at least one composition (B).

Another embodiment of the invention is a dyeing process comprising applying on keratin fibers, such as hair, a composition, which is ready to use, extemporarily prepared at the moment of use, leaving said dyeing composition to act for time, such as a resting time, ranging from 1 to 60 minutes approximately, such as from 10 to 45 minutes approximately, rinsing said keratin fibers, optionally shampooing said keratin fibers, again rinsing after said optional shampooing and drying said keratin fibers, wherein said composition comprises:

at least one colorant chosen from oxidation dyes and direct dyes, wherein said oxidation dyes are chosen from bases, couplers, and bases and couplers, and

at least one oxidizing agent, and

at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100. This process can be performed on keratin fibers, such as hair, that are either dry or wet.

Another process comprises applying on keratin fibers, such as hair, a composition, leaving said composition on said keratin fibers for a resting time ranging from 1 to 60 minutes approximately, such as from 10 to 45 minutes approximately, rinsing said keratin fibers, optionally shampooing said keratin fibers, and after said optional shampooing again rinsing and drying said keratin fibers, wherein said composition comprises:

at least one composition (A′) comprising at least one colorant chosen from oxidation dyes and direct dyes, wherein said oxidation dyes are chosen from bases, couplers, and bases and couplers,

at least one composition (A″) comprising at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100,

at least one oxidizing composition (B)

wherein said composition comprising said at least one compositions (A′), (A″) and (B) is prepared by mixing said at least one compositions (A′), (A″), and (B) at the moment of use.

The invention provides a dyeing device or “kit” having a plurality of compartments.

One aspect of the invention relates to a device comprising 2 compartments wherein:

a first compartment comprises at least one composition A1 comprising, in a medium appropriate for dyeing, at least one colorant chosen from oxidation dyes and direct dyes, wherein said oxidation dyes are chosen from bases, couplers, and bases and couplers, and wherein

a second compartment comprises at least one composition B1 comprising, in a medium appropriate for dyeing, at least one oxidizing agent, and wherein

said device comprises, in at least one composition A1, in at least one composition B1 or in at least one composition A1 and in at least one composition B1, at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and wherein n is an integer ranging from 1 to 100.

Another embodiment entails a device comprising 3 compartments wherein:

a first compartment comprises at least one composition A2 comprising, in a medium appropriate for dyeing, at least one colorant chosen from oxidation dyes and direct dyes, wherein said oxidation dyes are chosen from bases, couplers, and bases and couplers, and wherein

a second compartment comprises at least one composition B2 comprising, in a medium appropriate for dyeing, at least one oxidizing agent, and wherein

a third compartment comprises at least one composition C comprising, optionally in a medium appropriate for dyeing, at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and wherein n is an integer ranging from 1 to 100, and wherein

said at least one composition A2, said at least one composition B2, and both said at least one composition A2 and said at least one composition B2 can also comprise said at least one nonionic compound.

The present invention also relates to the use of the above-defined dyeing compositions or dyeing device or “kit” having a plurality of compartments for dyeing keratin fibers such as human hair.

As used herein, the term “lower alkyl” means a C₁-C₆ alkyl.

The at least one nonionic compound according to the present invention can be for example a compound of the formula R—(OCH₂CH₂)ₙ—OR′ wherein R is chosen from C₁₆-C₁₈ alkyl groups, R′ is chosen from C₁₆-C₁₈ alkyl groups optionally substituted with a hydroxyl group, and n is an integer equal to or larger than 20.

The at least one nonionic compound can be for example a compound of the formula R—(OCH₂CH₂)ₙ—OR′ wherein R and R′, which can be identical or different, are each chosen from C₁₆-C₁₈ alkyl groups, and n is equal to 4. Such a compound is for example described in the CTFA dictionary under the name of PEG 4 DITALLOW ETHER and can be prepared by reacting polyethylene glycol 4 ethylene oxide with a mixture of fatty alcohols, including the fatty alcohol derived from tallow fatty acid.

The at least one nonionic compound can be for example a compound of the formula R—(OCH₂CH₂)ₙ—OR′ wherein R is chosen from C₁₆-C₁₈ alkyl groups, R′ is chosen from substituted alkyl groups —C₁₄OH, and n is equal to 60. Such a compound is for example found in the CTFA dictionary under the names of CETEARETH 60 MYRISTYL GLYCOL and HYDROGENATED TALLOWETH 60 MYRISTYL GLYCOL. CETEARETH 60 MYRISTYL GLYCOL is for example sold by the company AKZO under the commercial name ELFACOS GT 282 S.

The at least one nonionic compound according to the invention is generally present in an amount approximately ranging from 0.05 wt % to 10 wt % of the total weight of the dyeing composition applied on the fibers, such as approximately from 0.1 wt % to 5 wt %.

The at least one oxidation dye which can be used according to the present invention is chosen from oxidation bases, and oxidation couplers. In one embodiment, the compositions can comprise at least one oxidation base.

The oxidation bases usable in the context of the present invention are chosen from those conventionally known as oxidation dyes. Representative oxidation dyes include paraphenylenediamines, double bases, ortho- and para-aminophenols and heterocyclic bases.

For example, the following oxidation bases may be used:

(I) para-phenylenediamines chosen from compounds of formula (I), and their acid addition salts:

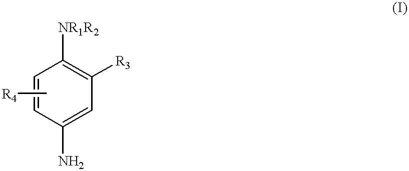

wherein:

R₁ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, phenyl groups, 4′-aminophenyl groups, and C₁-C₄ alkyl groups substituted with at least one group chosen from nitrogen-containing groups,

R₂ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, and C₁-C₄ alkyl groups substituted with a nitrogen-containing group;

R₁ and R₂ may also form, together with the nitrogen atom to which they are bonded, a 5- or 6-membered nitrogen-containing heterocycle ring, optionally substituted with at least one group chosen from alkyl groups, hydroxyl groups and ureido groups;

R₃ is chosen from hydrogen, halogens, such as chlorine, bromine, iodine and fluorine, C₁-C₄ alkyl groups, hydroxy(C₁-C₄ alkyl) groups, hydroxy(C₁-C₄ alkoxy) groups, acetylamino(C₁-C₄ alkoxy) groups, mesylamino(C₁-C₄ alkoxy) groups, and carbamoylamino(C₁-C₄ alkoxy) groups;

R₄ is chosen from hydrogen, halogens, and C₁-C₄ alkyl groups.

Suitable nitrogen-containing groups of formula (I) above may, for example, be chosen from amino, (C₁-C₄)mono alkylamino, (C₁-C₄)dialkylamino, (C₁-C₄)trialkylamino, monohydroxy(C₁-C₄)alkylamino, imidazolinium, and ammonium groups.

Representative para-phenylenediamines of formula (I) above which may be used include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-3-methylaniline, 4-amino-3-chloro-N,N-bis(β-hydroxyethyl)-aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, and their acid addition salts.

In other embodiments of the present invention, para-phenylenediamines of formula (I) above can, for example, be chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and their acid addition salts.

(II) double bases chosen from compounds comprising at least two aromatic rings substituted with at least one group chosen from amino and hydroxyl groups. Such double bases may be chosen from compounds of formula (II), and their acid addition salts:

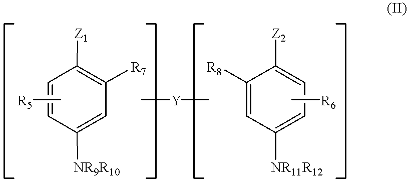

wherein:

Z₁ and Z₂, which may be identical or different, are each chosen from hydroxyl groups, and —NH₂ groups, optionally substituted with a group chosen from C₁-C₄ alkyl groups, and linkers Y;

linker Y is chosen from linear and branched, divalent alkylene groups comprising from 1 to 14 carbon atoms, optionally interrupted by, or optionally terminating with, at least one entity chosen from nitrogen-containing groups and heteroatoms such as oxygen, sulfur, and nitrogen, and optionally substituted with at least one group chosen from hydroxyl groups, and C₁-C₆ alkoxy groups;

R₅ and R₆, which may be identical or different, are each chosen from hydrogen, halogens, C₁-C₄ alkyl groups, monohydroxy(C₁-C₄ alkyl) groups, polyhydroxy(C₂-C₄ alkyl) groups, amino(C₁-C₄ alkyl) groups, and linkers Y;

R₇, R₈, R₉, R₁₀, R₁₁ and R₁₂, which may be identical or different, are each chosen from hydrogen, linkers Y, and C₁-C₄ alkyl groups;

provided that said compounds of formula (II) comprise only one linker Y per molecule.

Suitable nitrogen-containing groups of formula (II) include mono(C₁-C₄)alkylamino, (C₁-C₄)dialkylamino, (C₁-C₄)trialkylamino, monohydroxy(C₁-C₄)alkylamino, imidazolinium and ammonium groups.

Representative double bases of formula (II) include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their acid addition salts.

In another embodiment of the invention, the double bases of formula (II) may be chosen from N,N′-bis(β-hydroxyethyl)-N,N′-bis(4′-aminophenyl)-1,3-diaminopropanol, 1,8-bis(2,5-diaminophenoxy)-3,5-dioxaoctane, and their acid addition salts.

(III) para-aminophenols chosen from compounds of formula (III), and their acid addition salts:

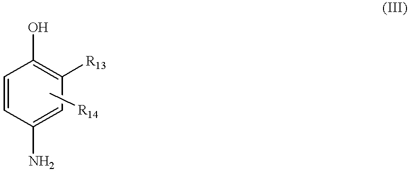

wherein:

R₁₃ is chosen from hydrogen, halogens, such as fluorine, C₁-C₄ alkyl groups, hydroxy(C₁-C₄ alkyl) groups, (C₁-C₄)alkoxy(C₁-C₄)alkyl groups, amino(C₁-C₄)alkylamino(C₁-C₄ alkyl) groups, and hydroxy(C₁-C₄)alkylamino(C₁-C₄ alkyl) groups;

R₁₄ is chosen from hydrogen, halogens, C₁-C₄ alkyl groups, monohydroxy(C₁-C₄ alkyl) groups, polyhydroxy(C₂-C₄ alkyl) groups, amino(C₁-C₄ alkyl) groups, cyano(C₁-C₄ alkyl) groups, and (C₁-C₄)alkoxy(C₁-C₄)alkyl groups;

it being understood that at least one of R₁₃ and R₁₄ is chosen from hydrogen.

Representative para-aminophenols of formula (III) above include para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and their acid addition salts.

(IV) ortho-aminophenols chosen, for example, from 2-aminophenol, 2-amino-1-hydroxy-5-methylbenzene, 2-amino-1-hydroxy-6-methylbenzene, 5-acetamido-2-aminophenol, and their acid addition salts.

(V) heterocyclic bases chosen, for example, from pyridine derivatives, pyrimidine derivatives, pyrazole derivatives, pyrazolo-pyrimidine derivatives, and their acid addition salts.

Representative pyridine derivatives include 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their acid addition salts. Some of the aforementioned pyridine derivatives have been described, for example in the patents GB 1,026,978 and GB 1,153,196, the disclosures of which are incorporated by reference herein.

Representative pyrimidine derivatives include 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and their acid addition salts. Some of the aforementioned pyrimidine derivatives have been described, for example in German Patent DE 2,359,399, Japanese Patents JP 88-169,571 and JP 91-333,495, and Patent Application WO 96/15765, the disclosures of which are incorporated by reference herein.

Representative pyrazolo-pyrimidine derivatives include those described, for example, in the patent application FR-A-2 750 048, the disclosure of which is incorporated by reference herein. Such pyrazolo-pyrimidine derivatives include pyrazolo[1,5-a]pyrimidines, such as

pyrazolo[1,5-a]pyrimidine-3,7-diamine;

2,5-dimethylpyrazolo[1,5-a]pyrimidine-3,7-diamine;

pyrazolo[1,5-a]pyrimidine-3,5-diamine;

2,7-dimethylpyrazolo[1,5-a]pyrimidine-3,5-diamine;

3-aminopyrazolo[1,5-a]pyrimidin-7-ol;

3-aminopyrazolo[1,5-a]pyrimidin-5-ol;

2-(3-aminopyrazolo[1,5-a]pyrimidin-7-ylamino)ethanol;

2-(7-aminopyrazolo[1,5-a]pyrimidin-3-ylamino)ethanol;

2-[(3-aminopyrazolo[1,5-a]pyrimidin-7-yl)-(2-hydroxyethyl)amino]ethanol;

2-[(7-aminopyrazolo[1,5-a]pyrimidin-3-yl)-(2-hydroxyethyl)amino]ethanol;

5,6-dimethylpyrazolo[1,5-a]pyrimidine-3,7-diamine;

2,6-dimethylpyrazolo[1,5-a]pyrimidine-3,7-diamine;

2,5,N7,N7-tetramethylpyrazolo[1,5-a]pyrimidine-3,7-diamine;

their salts, such as their acid addition salts, and their tautomeric forms when a tautomeric equilibrium exists.

Representative pyrazole derivatives include 4,5-diamino-1-methylpyrazole, 4,5-diamino-1-hydroxyethylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their acid addition salts. Some of the aforementioned pyrazole derivatives have been described, for example in Patents DE 3,843,892, DE 4,133,957 and Patent Applications WO 94/08969, WO 94/08970, FR-A-2,733,749 and DE 195 43 988, the disclosures of which are incorporated by reference herein.

In accordance with the present invention, the oxidation bases are generally present in an amount approximately ranging from 0.0005% to 12% by weight, relative to the total weight of said at least one composition (A).

Suitable couplers which may be used in the dyeing process of the invention include couplers conventionally used in oxidation dyeing compositions. Such couplers can be chosen, for example, from meta-phenylenediamines, meta-aminophenols, meta-diphenols, mono- and poly-hydroxylated derivatives of naphthalene, sesamol and its derivatives and heterocyclic couplers, such as, for example, indole couplers, indoline couplers, and pyridine couplers, and their acid addition salts.

Representative couplers include 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, β-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and their acid addition salts.

When these couplers are present, they are generally present in an amount approximately ranging from 0.0001% to 10% by weight, relative to the total weight of said at least one composition (A), such as approximately from 0.005% to 5%.

Generally, the acid addition salts of the oxidation bases and couplers can be chosen from hydrochlorides, hydrobromides, sulfates, tartrates, lactates and acetates.

Representative direct dyes which can be used in the present invention include direct dyes that have conventionally been used in direct dyeing compositions and lighting direct dyeing compositions. For example, the dyes can be chosen from nitrobenzene, anthraquinone, azo, neutral, cationic and anionic dyes, naphthoquinone dyes, triarylmethane dyes, xanthene dyes, Arianors and dyes described in the patent applications WO 95/01772, WO 95/15144 and EP-A-0 714 954, the disclosures of which are incorporated by reference herein. Generally, the direct dyes are used in amounts approximately ranging from 0.01% to 20% by weight of the total weight of the composition.

In one embodiment of the invention, said at least one composition (A) and said at least one composition (B) can further comprise at least one polymer chosen from cationic and amphoteric polymers, such as substantive polymers.

Representative cationic polymers which may be used in accordance with the present invention include any of those already known to improve at least one cosmetic property of hair, such as, for example, those described in patent application EP-A-0 337 354 and in French patent applications FR-A-2 270 846, 2 383 660, 2 598 611, 2 470 596 and 2 519 863, the disclosures of which are incorporated herein by reference.

As used herein, “cationic polymer” refers to polymers chosen from polymers comprising at least one cationic group and polymers comprising at least one group which can be ionized to form cationic groups.

According to the present invention, the at least one cationic polymer may be chosen from polymers comprising at least one unit, wherein said at least one unit comprises at least one group chosen from primary amine groups, secondary amine groups, tertiary amine groups and quaternary amine groups, wherein said at least one group forms part of the polymer skeleton, or is carried by at least one lateral substituent on said polymer skeleton.

According to the present invention, the at least one cationic polymer has a number-average molecular mass generally ranging from 500 approximately to 5×10⁶ approximately, such as from 1×10³ approximately to 3×10⁶ approximately.

The at least one cationic polymer may, for example, be chosen from polymers of quaternary polyammonium type, polymers of polyamino amide type and polymers of polyamine type. Such types of polymers are known in the art. They are for example described in French patents Nos. 2,505,348 and 2,542,997, the disclosures of which are incorporated by reference herein.

Non-limiting examples of cationic polymers include:

(1) homo- and co-polymers derived from at least one monomer chosen from acrylic esters, methacrylic esters and amides, wherein said homo- and co-polymers comprise at least one unit chosen from units of formulae:

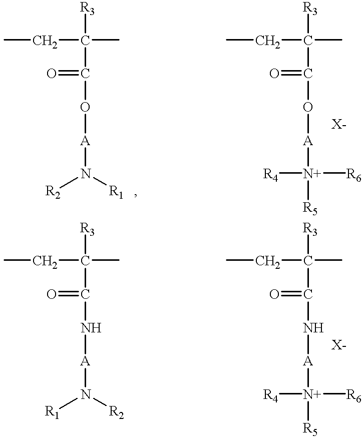

wherein:

R₃, which may be identical or different, are each chosen from hydrogen atoms and CH₃ groups;

A, which may be identical or different, are each chosen from linear and branched alkyl groups comprising from 1 to 6 carbon atoms, such as 2 and 3 carbon atoms, and hydroxyalkyl groups comprising from 1 to 4 carbon atoms;

R₄, R₅ and R₆, which may be identical or different, are each chosen from alkyl groups comprising from 1 to 18 carbon atoms, such as from 1 to 6 carbon atoms, and benzyl groups;

R₁ and R₂, which may be identical or different, are each chosen from hydrogen atoms and alkyl groups comprising from 1 to 6 carbon atoms, such as methyl and ethyl;

X⁻ is an anion chosen from anions derived from at least one inorganic acid and anions derived from at least one organic acid, such as methosulfate anions and halides, such as chlorides and bromides.

Copolymers of family (1) may further comprise at least one unit derived from at least one comonomer chosen from vinyllactams, vinyl esters, acrylamides, methacrylamides, diacetone acrylamides, acrylamides and methacrylamides substituted on the nitrogen with at least one group chosen from (C₁-C₄) alkyls, acrylic acids, methacrylic acids, acrylic esters, and methacrylic esters. Non-limiting examples of vinyllactams include vinylpyrrolidone and vinylcaprolactam.

Non-limiting examples of copolymers of family (1) include:

copolymers derived from at least one monomer of (i) acrylamide and (ii) dimethylaminoethyl methacrylate quaternized with at least one group chosen from dimethyl sulfate and dimethyl halide, such as the product sold under the name HERCOFLOC by the company Hercules;

copolymers derived from at least one monomer of (i) acrylamide and (ii) methacryloyloxyethyltrimethylammonium chloride described, for example, in patent application EP-A-080 976, the disclosure of which is incorporated herein by reference, and which is sold under the name BINA QUAT P 100 by the company Ciba Geigy;

copolymers derived from at least one monomer of (i) acrylamide and (ii) methacryloyloxyethyltrimethylammonium methosulfate, such as, for example, copolymers sold under the name RETEN by the company Hercules;

quaternized and non-quaternized vinylpyrrolidone/dialkylaminoalkyl acrylate copolymers and quaternized and non-quaternized vinylpyrrolidone/dialkylaminoalkyl methacrylate copolymers, such as the products sold under the name “GAFQUAT” by the company ISP, such as, for example, “GAFQUAT 734” or “GAFQUAT 755” and the products known as “COPOLYMER 845, 958 and 937”. These polymers are described in detail in French patents 2 077 143 and 2 393 573, the disclosures of which are incorporated herein by reference;

dimethylaminoethyl methacrylate/vinylcaprolactam/vinylpyrrolidone terpolymers, such as the product sold under the name GAFFIX VC 713 by the company ISP;

vinylpyrrolidone/methacrylamidopropyldimethylamine copolymers, such as the product sold under the name STYLEZE CC 10 by ISP; and

quaternized vinylpyrrolidone/dimethylaminopropylmethacrylamide copolymers, such as the product sold under the name “GAFQUAT HS 100” by the company ISP;

(2) cellulose ether derivatives comprising quaternary ammonium groups, such as those described in French patent 1,492,597, the disclosure of which is incorporated herein by reference, and polymers sold under the names “JR” (JR 400, JR 125 and JR 30M) and “LR” (LR 400, or LR 30M) by the company Union Carbide Corporation. These polymers are also defined in the CTFA dictionary as quaternary ammoniums of hydroxyethylcellulose which have reacted with an epoxide substituted with a trimethylammonium group;

(3) cationic cellulose derivatives such as cellulose copolymers and cellulose derivatives grafted with at least one water-soluble monomer of quaternary ammonium, such as those described in U.S. Pat. No. 4,131,576, the disclosure of which is incorporated herein by reference, such as hydroxyalkylcelluloses (such as, for example, hydroxymethylcelluloses, hydroxyethylcelluloses and hydroxypropylcelluloses, wherein said hydroxyalkylcelluloses are grafted with at least one salt chosen from, for example, methacryloylethyltrimethylammonium salts, methacrylamidopropyltrimethylammonium salts and dimethyldiallylammonium salts). For example, commercial products corresponding to the aforementioned cationic cellulose derivatives include the products sold under the names “CELQUAT L 200” and “CELQUAT H 100” by the company National Starch;

(4) cationic polysaccharides, such as those described in U.S. Pat. Nos. 3,589,578 and 4,031,307, the disclosures of which are incorporated herein by reference, such as guar gums comprising at least one cationic trialkylammonium group. For example, guar gums modified with at least one salt, such as a chloride salt, of 2,3-epoxypropyltrimethylammonium may be used in the present invention. Such products are sold in particular under the trade names JAGUAR C13 S, JAGUAR C 15, JAGUAR C 17 and JAGUAR C162 by the company Meyhall.

(5) polymers comprising (i) at least one piperazinyl unit and (ii) at least one group chosen from divalent alkylene groups and divalent hydroxyalkylene groups, wherein said at least one group optionally comprises at least one chain chosen from straight chains and branched chains, wherein said at least one chain is optionally interrupted by at least one entity chosen from oxygen atoms, sulfur atoms, nitrogen atoms, aromatic rings and heterocyclic rings, the oxidation products of said polymers and the quaternization products of said polymers. For example, such polymers are described in French patents 2,162,025 and 2,280,361, the disclosures of which are incorporated herein by reference;

(6) water-soluble polyamino amides which may be prepared by at least one polycondensation reaction of at least one acidic compound and at least one polyamine compound, wherein said polyamino amides may be crosslinked with at least one crosslinking agent chosen from epihalohydrins, diepoxides, dianhydrides, unsaturated dianhydrides, bis-unsaturated derivatives, bis-halohydrins, bis-azetidiniums, bis-haloacyldiamines, bis-alkyl halides and oligomers derived from reaction of at least one difunctional compound with at least one compound chosen from bis-halohydrins, bis-azetidiniums, bis-haloacyidiamines, bis-alkyl halides, epihalohydrins, diepoxides and bis-unsaturated derivatives, wherein said crosslinking agent may be used in a proportion generally ranging from 0.025 mol to 0.35 mol per amine group of said polyamino amide, wherein said polyamino amides may optionally be alkylated, and wherein if said polyamino amides comprise at least one tertiary amine group, said polyamino amides may optionally be quaternized. For example, such polymers are described in French patents 2,252,840 and 2,368,508, the disclosures of which are incorporated herein by reference;

(7) polyamino amide derivatives derived from condensation of at least one polyalkylene polyamine with at least one polycarboxylic acid, followed by alkylation with at least one bifunctional agent. Non-limiting examples of such polyamino amide derivatives include adipic acid/dialkylaminohydroxyalkyldialkylenetriamine polymers wherein the alkyl group comprises from 1 to 4 carbon atoms, such as methyl groups, ethyl groups and propyl groups. For example, such polymers are described in French patent 1,583,363, the disclosure of which is incorporated herein by reference.

Other non-limiting examples of such derivatives include the adipic acid/dimethylaminohydroxypropyl/diethylenetriamine polymers sold under the name “CARTARETINE F, F4 or F8” by the company Sandoz.

(8) polymers derived from the reaction of (i) at least one polyalkylene polyamine comprising two primary amine groups and at least one secondary amine group with (ii) at least one dicarboxylic acid chosen from diglycolic acid and saturated aliphatic dicarboxylic acids comprising from 3 to 8 carbon atoms. According to the present invention, the molar ratio of the at least one polyalkylene polyamine to the at least one dicarboxylic acid generally ranges from 0.8:1 to 1.4:1. The polyamino amide resulting from the above reaction may be reacted with epichlorohydrin in a molar ratio of epichlorohydrin to the at least one secondary amine group of the polyamino amide generally ranging from 0.5:1 to 1.8:1. For example, such polymers are described in U.S. Pat. Nos. 3,227,615 and 2,961,347, the disclosures of which are incorporated herein by reference.

Polymers of this type are sold in particular under the name “HERCOSETT 57” by the company Hercules Inc. and under the name “PD 170” or “DELSETTE 101” by the company Hercules in the case of adipic acid/epoxypropyl/diethylenetriamine copolymers.

(9) cyclopolymers of alkyldiallylamine and cyclopolymers of dialkyldiallylammonium, such as homopolymers and copolymers comprising, as the main constituent of the chain, at least one unit chosen from units of formulae (VI) and (VI′):

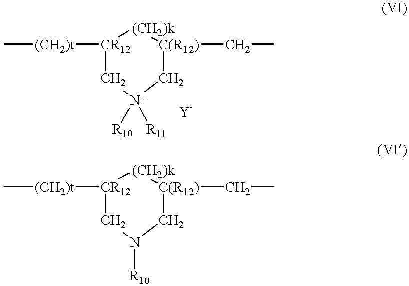

wherein:

k and t, which may be identical or different, are each chosen from 0 and 1, with the proviso that the sum of k+t is equal to 1;

R₁₂, which may be identical or different, are each chosen from hydrogen atoms and methyl groups;

R₁₀ and R₁₁, which may be identical or different, are each chosen from alkyl groups comprising from 1 to 22 carbon atoms, hydroxyalkyl groups wherein alkyl group optionally comprises from 1 to 5 carbon atoms, and C₁-C₄ amidoalkyl groups;

R₁₀ and R₁₁, together with the nitrogen atom to which they are commonly bonded, form at least one heterocyclic group, such as piperidyl groups and morpholinyl groups;

Y⁻ is an anion, such as bromide, chloride, acetate, borate, citrate, tartrate, bisulfate, bisulfite, sulfate and phosphate. For example, such polymers are described in French patent 2,080,759 and in its Certificate of Addition 2,190,406, the disclosures of which are incorporated herein by reference.

Non-limiting examples of the polymers defined above include the dimethyldiallylammonium chloride homopolymer sold under the name “MERQUAT 100” by the company Calgon (and its homologues of low weight-average molecular mass) and copolymers of diallyldimethylammonium chloride and of acrylamide, sold under the name “MERQUAT 550”.

(10) quaternary diammonium polymers comprising at least two repeating units of formula (VII):

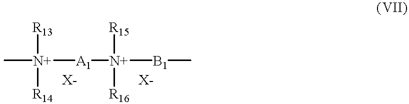

wherein:

R₁₃, R₁₄, R₁₅ and R₁₆, which may be identical or different, are each chosen from aliphatic groups comprising from 1 to 20 carbon atoms, alicyclic groups comprising from 3 to 20 carbon atoms, arylaliphatic groups comprising from 5 to 20 carbon atoms, and lower hydroxyalkyl groups; and

additionally at least two of said R₁₃, R₁₄, R₁₅ and R₁₆, together with the nitrogen atoms to which they are attached, may form at least one heterocycle optionally comprising an additional heteroatom other than nitrogen; and

additionally, R₁₃, R₁₄, R₁₅ and R₁₆, which may be identical or different, may each be chosen from linear and branched C₁-C₆ alkyl groups substituted with at least one group chosen from nitrile groups, ester groups, acyl groups, amide groups and groups chosen from groups of formulae —CO—O—R₁₇-D and —CO—NH—R₁₇-D wherein R₁₇ is chosen from alkylene groups and D is chosen from quaternary ammonium groups;

A₁ and B₁, which may be identical or different, are each chosen from polymethylene groups comprising from 2 to 20 carbon atoms, chosen from linear and branched, saturated and unsaturated polymethylene groups wherein said polymethylene groups may optionally comprise, optionally linked to and optionally intercalated in the main chain, at least one entity chosen from aromatic rings, oxygen atoms, sulfur atoms, sulfoxide groups, sulfone groups, disulfide groups, amino groups, alkylamino groups, hydroxyl groups, quaternary ammonium groups, ureido groups, amide groups and ester groups; and

X⁻ is an anion chosen from anions derived from inorganic acids and anions derived from organic acids; and

A₁, R₁₃ and R₁₅ may optionally form, together with the nitrogen atoms to which they are attached, at least one piperazine ring;

with the proviso that if A₁ is chosen from linear and branched, saturated and unsaturated alkylene groups and linear and branched, saturated and unsaturated hydroxyalkylene groups, B₁ may also be chosen from groups of formula:

(CH₂)ₙ—CO-D-OC—(CH₂)ₙ—

wherein:

n is a number such that the overall quaternary diammonium polymer has a number-average molecular weight ranging from 1000 to 100,000;

D is chosen from:

a) glycol residues of formula: —O-Z-O—, wherein Z is chosen from linear and branched hydrocarbon groups and groups chosen from groups of formulae:

—(CH₂—CH₂—O)ₓ—CH₂—CH₂—; and

—[CH₂—CH(CH₃)—O]_{y}—CH₂—CH(CH₃)—

wherein x and y, which may be identical or different, are each chosen from integers ranging from 1 to 4 (in which case x and y represent a defined and unique degree of polymerization) and any number ranging from 1 to 4 (in which case x and y represent an average degree of polymerization);

b) bis-secondary diamine residues such as piperazine derivatives;

c) bis-primary diamine residues chosen from residues of formula: —NH—Y—NH—, wherein Y is chosen from linear and branched hydrocarbon groups and residues of formula

—CH₂—CH₂—S—S—CH₂—CH₂—; and

d) ureylene groups of formula: —NH—CO—NH—.

In one embodiment, X⁻ is an anion chosen from chloride ions and bromide ions.

According to the present invention, the quarternary diammonium polymers have a number-average molecular mass generally ranging from 1000 to 100,000.

For example, polymers of this type are described in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020, the disclosures of which are incorporated herein by reference.

Further, according to the present invention, polymers comprising at least two repeating units of formula (a) may be used:

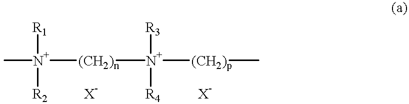

wherein:

R₁, R₂, R₃ and R₄, which may be identical or different, are each chosen from alkyl groups comprising from 1 to 4 carbon atoms and hydroxyalkyl groups comprising from 1 to 4 carbon atoms;

n and p, which may be identical or different, are each chosen from integers ranging from 2 to 20; and

X⁻ is an anion chosen from anions derived from inorganic acids and anions derived from organic acids.

(11) polyquaternary ammonium polymers comprising at least one unit of formula (VIII):

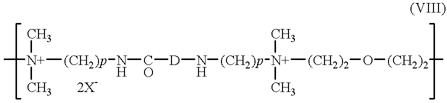

wherein:

p is an integer ranging from 1 to 6,

D is chosen from direct bonds and —(CH₂)ᵣ—CO— groups, wherein r is a number equal to 4 or 7, and

X⁻ is an anion chosen from anions derived from inorganic acids and anions derived from organic acids.

For example, such compounds are described in patent application EP-A-122,324, the disclosure of which is incorporated by reference herein, and may be prepared according to the procedures described in the U.S. Pat. Nos. 4,157,388, 4,390,689, 4,702,906, and 4,719,282, the disclosures of which are incorporated by reference herein.

Non-limiting examples of the polyquarternary ammonium polymers are those having a molecular weight, measured by NMR of Carbon 13, less than 100000, and, wherein in formula (VIII), p is equal to 3, and wherein

a) D is a —(CH₂)₄—CO— group, X⁻ is a chloride, the molecular weight measured by NMR of Carbon 13 (NMR¹³C) is approximately 5600; this type of polymer is available from the company MIRANOL under the name MIRAPOL-AD1;

b) D is a —(CH₂)₇—CO— group, X⁻ is a chloride, the molecular weight measured by NMR of Carbon 13 (NMR¹³C) is approximately 8100; this type of polymer is available from the company MIRANOL under the name MIRAPOL-AZ1;

c) D is a direct bond, X⁻ is a chloride, the molecular weight measured by NMR of Carbon 13 (NMR¹³C) is approximately 25500; this type of polymer is available from the company MIRANOL under the name MIRAPOL-A15;

d) a “Block Copolymer” formed of the units corresponding to the polymers described in paragraphs a) and c), available from the company MIRANOL under the names NIRAPOL-9, (molecular weight NMR¹³C, approximately 7800) MIRAPOL-175, (molecular weight NMR¹³C, approximately 8000) and MIRAPOL-95, (molecular weight NMR¹³C, approximately 12500).

In one embodiment of the invention, the polymer of the unit of formula (VIII) can be a polymer where p is equal to 3, D is a direct bond, X⁻ is a chloride, the molecular weight measured by NMR of Carbon 13, (NMR13C) is approximately 25500.

(12) quaternary polymers of vinylpyrrolidone and quaternary polymers of vinylimidazole, such as, for example, the products sold under the names LUVIQUAT FC 905, FC 550 and FC 370 by the company BASF.

(13) polyamines, such as POLYQUART H sold by Henkel under the reference name “POLYETHYLENE GLYCOL (15) TALLOW POLYAMINE” in the CTFA dictionary.

(14) crosslinked (meth)acryloyloxy(C₁-C₄)alkyltri(C₁-C₄)alkylammonium salt polymers, such as the polymers derived from homopolymerization of dimethylaminoethyl methacrylate quaternized with methyl chloride and polymers derived from copolymerization, for example, of acrylamide with dimethylaminoethyl methacrylate quaternized with a methyl halide (such as methyl chloride), wherein the homo- or copolymerization is followed by crosslinking with at least one compound comprising olefinic unsaturation, such as methylenebisacrylamide. For example, a crosslinked acrylamide/methacryloyloxyethyltrimethylammonium chloride copolymer (20/80 by weight) in the form of a dispersion comprising about 50% by weight of said copolymer in mineral oil may be used. This dispersion is sold under the name “SALCARE SC 92” by the company Allied Colloids. Further, a crosslinked methacryloyloxyethyltrimethylammonium chloride homopolymer comprising about 50% by weight of the homopolymer in mineral oil or in a liquid ester may be used. These dispersions are sold under the names “SALCARE SC 95” and “SALCARE SC 96” by the company Allied Colloids.

Other cationic polymers which may be used as the at least one cationic polymer according to the present invention are polyalkyleneimines (such as polyethyleneimines), polymers comprising at least one vinylpyridine unit, polymers comprising at least one vinylpyridinium unit, condensates of polyamines, condensates of epichlorohydrin, quaternary polyureylenes and chitin derivatives.

Other embodiments of the invention use cationic polymers chosen from the polymers of (1), (9), (10), (11) and (14). Specifically, polymers of formulae (W) and (U) can be used:

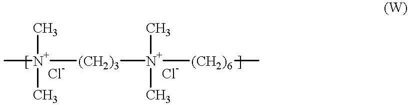

such as those of which the molecular weight, determined by gel chromatography, ranges from 9500 to 9900;

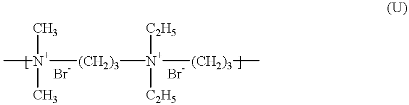

and in particular those of which the molecular weight, determined by gel chromatography, is approximately 1200.

Generally, the cationic polymers are present in an amount approximately ranging from 0.01% to 10% by weight, such as approximately from 0.05% to 5% by weight and further such as approximately from 0.1% to 3% by weight, relative to the total weight of the final composition.

The amphoteric polymers which can be used in the present invention can be chosen from polymers comprising K and M units distributed statistically in the polymer chain, wherein:

K is a unit derived from a monomer comprising at least one basic nitrogen atom and M is a unit derived from an acid monomer comprising at least one group chosen from carboxylic groups and sulfonic groups; or alternatively

K and M, which are identical or different, are each groups chosen from groups derived from zwitterionic monomers of carboxybetaines and groups derived from zwitterionic monomers of sulfobetaines; or alternatively

K and M, which are identical or different, are each chosen from polymers comprising cationic polymer chains comprising at least one amine group chosen from primary, secondary, tertiary and quaternary amine groups, wherein at least one of the amine groups is substituted with a group chosen from carboxylic groups and sulphonic groups linked via a hydrocarbon radical; or alternatively

K and M form part of a chain of a polymer with an α,β-dicarboxylic ethylene unit wherein one of the carboxylic groups has been caused to react with a polyamine comprising at least one amine group chosen from primary amine groups and secondary amine groups.

Representative of the film forming amphoteric polymers defined above that can be used include the following polymers:

(1) The polymers resulting from the copolymerization of a monomer derived from a vinyl compound substituted with a carboxylic group such as acrylic acid, methacrylic acid, maleic acid, and α-chloroacrylic acid, and of a basic monomer derived from a substituted vinyl compound comprising at least one basic atom such as dialkylaminoalkyl methacrylate and acrylate, dialkylaminoalkylmethacrylamide and acrylamide. Such compounds are described in U.S. Pat. No. 3,836,537, the disclosure of which is incorporated by reference herein. Copolymer of the sodium acrylate/acrylamidopropyl-trimethylammonium chloride sold under the name of “POLYQUART KE 3033” by the company HENKEL can also be cited.

The vinyl compound can also be a salt of dialkyldiallylammonium such as diethyldiallylammonium chloride. The copolymers of acrylic acid and the latter monomer are proposed under the name “MERQUAT 280”, “MERQUAT 295” and “MERQUAT PLUS 3330” by the company CALGON.

(2) The polymers comprising units derived from:

a) at least one monomer chosen from acrylamides substituted on the nitrogen by an alkyl radical and methacrylamides substituted on the nitrogen by an alkyl radical,

b) at least one acidic comonomer comprising at least one reactive carboxylic group, and

c) at least one basic comonomer such as comonomers chosen from esters of acrylic acid and esters of methacrylic acid, said esters being substituted with at least one amine chosen from primary, secondary, tertiary and quaternary amines, and the product of quaternization of dimethylaminoethyl methacrylate with a sulfate chosen from dimethyl sulfate and diethyl sulfate.

Some embodiments according to the invention utilize n-substituted acrylamides and methacrylamides comprising (C₂-C₁₂)alkyl groups, such as N-ethylacrylamide, N-tert-butylacrylamide, N-tert-octylacrylamide, N-octylacrylamide, N-decylacrylamide, and N-dodecylacrylamide, as well as the corresponding methacrylamides.

The acidic comonomers can be chosen, for example, from acrylic, methacrylic, crotonic, itaconic, maleic and fumaric acids as well as the (C₁-C₄)alkyl monoesters of entities chosen from maleic anhydride, fumaric anhydride, maleic acid, and fumaric acid.

The basic comonomers can be chosen, for example, from methacrylates of aminoethyl, butylaminoethyl, N,N′-dimethylaminoethyl, and N-tert-butylaminoethyl.

Additionally, the copolymers having the CTFA name (4^{th} edition, 1991) Octylacrylamide/acrylates/butylaminoethylmethacrylate copolymer such as the products sold under the name AMPHOMER and LOVOCRYL 47 by the company NATIONAL STARCH can also be used.

(3) The partially and completely alkylated and crosslinked polyaminoamides derived from polyaminoamides of formula:

[CO—R₂₇—CO-Z] (VIII)

wherein:

R₂₇ is a divalent group chosen from groups derived from saturated dicarboxylic acids, groups derived from dicarboxylic aromatic acids, groups derived from mono- and dicarbocylic aliphatic acids comprising at least one ethylenic double bond, groups derived from an ester of (C₁-C₆)alkanols of said acids, and groups derived from the addition of any one of said aforementioned acids with an amine chosen from bis-primary and bis-secondary amines, and

Z is a divalent group derived from polyalkylene-polyamines chosen from bis-primary, mono- and bis-secondary polyalkylene-polyamines, for example, Z represents

a) in an amount approximately ranging from 60 mol % to 100 mol %, the group

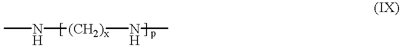

wherein x=2 and p=2 or 3, or alternatively x=3 and p=2,

it being understood that group Z of formula a) is derived from a compound chosen from diethylenetriamine, triethylenetetraamine and dipropylenetriamine;

b) in an amount approximately ranging from 0 mol % to 40 mol %, (1) said groups (IX) above in which x=2 and p=1 and which said group is derived from a compound chosen from ethylenediamine, and (2) groups derived from piperazine:

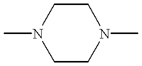

c) in an amount approximately ranging from 0 mol % to 20 mol %, the polyalkylene-polyamine group —NH—(CH₂)₆—NH—, which is derived from hexamethylenediamine, wherein said polyalkylene-polyamine group is crosslinked by adding a bifunctional crosslinking agent (chosen from the epihalohydrins, diepoxides, dianhydrides, and bis-unsaturated derivatives) present in an amount ranging from 0.025 mol to 0.35 mol of crosslinking agent per amine group of the polyamino amide and alkylated by the action of at least one compound chosen from acrylic acid, chloroacetic acid, alkanesultones, and salts of said alkanesltones.

The saturated dicarboxylic acids are for example chosen from saturated (C₆-C₁₀) dicarboxylic acids such as adipic, 2,2,4-trimethyladipic and 2,4,4-trimethyladipic acid. Representative dicarboxylic aromatic acids include for example (C₆-C₁₀) dicarboxylic aromatic acids, such as terephthalic acid. And representative mono- and dicarboxylic aliphatic acids comprising at least one ethylenic double bond include for example acrylic, methacrylic and itaconic acids.

The alkanesultones used in the alkylation can, for example, be chosen from propanesultone and butanesultone, and the salts of the alkylating agents can be chosen from sodium and potassium salts of said alkylating agents.

(4) The polymers comprising zwitterionic units of formula:

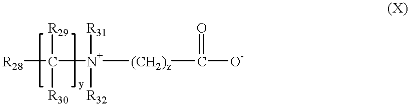

wherein:

R₂₈ is chosen from polymerizable unsaturated groups such as acrylate, methacrylate, acrylamide and methacrylamide groups,

y and z, which can be identical or different, are each chosen from integers ranging from 1 to 3,

R₂₉ and R₃₀, which may be identical or different, are each chosen from hydrogen, and methyl, ethyl and propyl groups,

R₃₁ and R₃₂, which may be identical or different, are each chosen from hydrogen and alkyl groups, provided that the sum of the carbon atoms in R₃₁ and R₃₂ does not exceed 10.

The polymers comprising such units may also comprise units derived from nonzwitterionic monomers such as dimethylaminoethyl acrylate, dimethylaminoethyl methacrylate, diethylaminoethyl acrylate, diethylaminoethyl methacrylate, alkyl acrylates, alkyl methacrylates, alkyl acrylamides, alkyl methacrylamides, and vinyl acetate.

By way of example, there may be mentioned the copolymer of methyl methacrylate/methyl dimethylcarboxymethylammonioethyl methacrylate such as the product sold under the name DIAFORMER Z301 by the company SANDOZ.

(5) The polymers derived from chitosan comprising at least one monomeric unit chosen from formulae (D), (E) and (F):

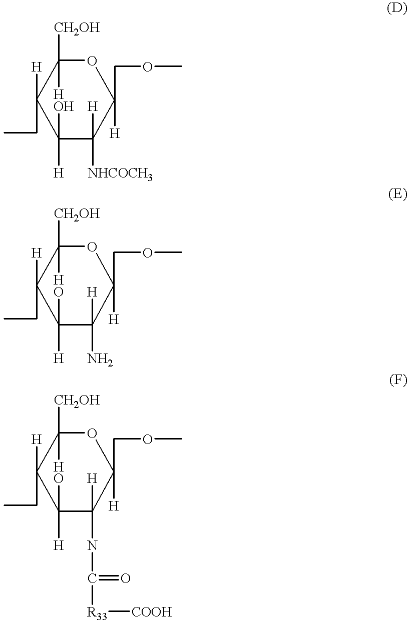

wherein said unit (D) is present in an amount approximately ranging from 0% to 30%, by weight relative to the total weight of said polymer, said unit (E) in an amount approximately ranging from 5% to 50%, by weight relative to the total weight of said polymer, and said unit (F) in an amount approximately ranging from 30% to 90%, by weight relative to the total weight of said polymer,

and wherein in said unit (F), R₃₃ is chosen from groups of formula:

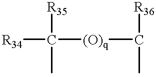

wherein:

q is equal to 0 or 1, and (i) when q is equal to 0, R₃₄, R₃₅ and R₃₆, which may be identical or different, are each chosen from:

hydrogen,

methyl, hydroxyl, acetoxy, and amino groups,

monoalkylamine and dialkylamine groups optionally interrupted by at least one nitrogen atom and/or optionally substituted with at least one group chosen from amine, hydroxyl, carboxyl, alkylthio and sulfonic groups, and

alkylthio groups wherein said alkyl portion of said alkylthio group carries an amino group,

provided that at least one of said R₃₄, R₃₅ and R₃₆ groups is chosen from hydrogen; and

(ii) when q is equal to 1, R₃₄, R₃₅ and R₃₆, which may be identical or different, are each chosen from hydrogen,

and the salts formed by these polymers (5) with bases, and the salts formed by these polymers (5) with acids.

(6) The polymers derived from the N-carboxyalkylation of chitosan such as N-carboxymethyl chitosan and N-carboxybutyl chitosan sold under the name “EVALSAN” by the company JAN DEKKER.

(7) The polymers of formula (XI), which are described for example in French Patent 1 400366, the disclosure of which is incorporated by reference herein:

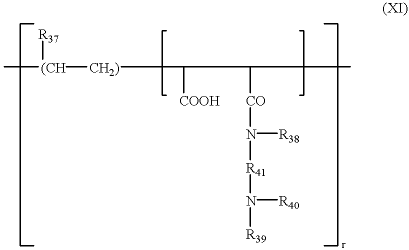

wherein:

r is chosen such that the number-average molecular weight of said polymer ranges from 500 to 6,000,000, such as from 1000 to 1,000,000.

R₃₇ is chosen from hydrogen and CH₃O, CH₃CH₂O, and phenyl groups,

R₃₈ and R₃₉, which are identical or different, are each chosen from hydrogen and lower alkyl groups such as methyl and ethyl,

R₄₀ is chosen from lower alkyl groups such as methyl and ethyl and groups of formula: —R₄₁—N(R₃₉)₂, comprising up to 6 carbon atoms, wherein R₃₉ is as defined above and R₄₁ is defined below,

R₄₁ is chosen from —CH₂—CH₂—, —CH₂—CH₂—CH₂—, and —CH₂—CH(CH₃)—.

(8) Amphoteric polymers of the -D-X-D-X- type chosen from:

a) polymers derived from reaction of chloroacetic acid or sodium chloroacetate with at least one compound comprising at least one unit of formula (XII):

-D-X-D-X-D- (XII)

wherein D is a group:

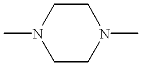

and X is chosen from the symbols E and E′, wherein E and E′, which are identical or different, are each chosen from bivalent groups chosen from alkylene groups comprising at least one chain chosen from linear and branched chains comprising up to 7 carbon atoms in the principal chain, wherein said principal chain is optionally substituted with at least one hydroxyl group, and wherein said principal chain optionally comprises at least one atom chosen from oxygen atoms, nitrogen atoms and sulfur atoms, wherein said at least one optional atom is present in the form of at least one group chosen from ether, thioether, sulfoxide, sulfone, sulfonium, alkylamine and alkenylamine groups, and hydroxyl, benzylamine, amine oxide, quaternary ammonium, amide, imide, alcohol, ester and urethane groups, and wherein said principal chain optionally comprises 1 to 3 rings chosen from aromatic rings and heterocyclic rings,

b) polymers of formula:

-D-X-D-X- (XIII)

wherein:

D is chosen from groups:

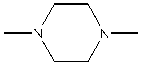

X is chosen from the symbols E and E′ and wherein at least one X is chosen from E′,

E is chosen from bivalent groups chosen from alkylene groups comprising at least one chain chosen from linear and branched chains comprising up to 7 carbon atoms in the principal chain, wherein said principal chain is optionally substituted with at least one hydroxyl group, and wherein said principal chain optionally comprises at least one atom chosen from oxygen atoms, nitrogen atoms, and sulfur atoms, wherein said at least one optional atom is present in the form of at least one group chosen from ether, thioether, sulfoxide, sulfone, sulfonium, alkylamine and alkenylamine groups, and hydroxyl, benzylamine, amine oxide, quaternary ammonium, amide, imide, alcohol, ester and urethane groups, and wherein said principal chain optionally comprises 1 to 3 rings chosen from aromatic rings and heterocyclic rings, and

E′ is a bivalent group chosen from alkylene groups comprising at least one chain chosen from linear and branched chains comprising up to 7 carbon atoms in the principal chain, wherein said principal chain is optionally substituted with at least one hydroxyl group and wherein said principal chain comprises at least one nitrogen atom substituted with an alkyl chain, wherein said alkyl chain is optionally interrupted by an oxygen atom and, wherein said alkyl chain comprises at least one functional group chosen from carboxyl and hydroxyl functional groups, and wherein said at least one alkyl chain is betainized by reaction with a reactant chosen from chloroacetic acid and sodium chloroacetate.

(9) The copolymers (C₁-C₅)alkyl vinyl ether/maleic anhydride partially modified by semiamidation with an N,N-dialkylaminoalkylamine such as N,N-dimethylaminopropylamine or by semiesterification with an N,N-dialkanolamine. These copolymers may also comprise other vinyl comonomers such as vinylcaprolactam.

The amphoteric polymers of family (1) are utilized in certain embodiments of the invention.

According to the invention, the at least one polymer chosen from cationic and amphoteric polymers may be present in an amount approximately ranging from 0.01% to 10% by weight, such as approximately from 0.05% to 5% by weight, and further such as approximately from 0.1% to 3% by weight of the total weight of the composition.

The composition according to the invention can also comprise at least one surfactant chosen from anionic, amphoteric, nonionic, zwitterionic and cationic surfactants.

Representative choices for the at least one surfactants include the following:

(i) Anionic Surfactant(s)

Representative anionic surfactants include salts (for example alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl(C₆-C₂₄) sulfosuccinates, alkyl(C₆-C₂₄) ether sulfosuccinates, alkyl(C₆-C₂₄)amide sulfosuccinates, alkylsulfosuccinamates alkyl(C₆-C₂₄) sulfoacetates, acyl(C₆-C₂₄) sarcosinates, acyl(C₆-C₂₄) glutamates, acyl isethionates, N-acyltaurates, and alkyl(C₆-C₂₄)polyglycoside carboxylic esters such as alkylglucoside citrates, alkylpolyglycoside tartrate and alkylpolyglycoside sulfosuccinates. The alkyl and acyl radicals of all of these various compounds can for example comprise from 12 to 20 carbon atoms, and the aryl radicals can for example be chosen from phenyl and benzyl groups.

For example, anionic surfactants can be chosen from fatty acid salts such as the salts of oleic acid, ricinoleic acid, palmitic acid, stearic acid, the acids of copra oil and the acids of hydrogenated copra oil, and acyl lactylates in which the acyl radical comprises from 8 to 20 carbon atoms. At least one weakly anionic surfactant can also be used, such as alkyl-D-galactosideuronic acids and their salts, as well as polyoxyalkylenated carboxylic (C₆-C₂₄)alkyl ether acids, polyoxyalkylenated carboxylic (C₆-C₂₄)alkylaryl ether acids, polyoxyalkylenated carboxylic (C₆-C₂₄)alkyl amidoether acids and their salts, for example, those comprising from 2 to 50 ethylene oxide groups.

(ii) Nonionic Surfactant(s)

Useful nonionic surfactants include compounds that are well known per se (see for example in this respect “Handbook of Surfactants” by M. R. Porter, published by Blackie & Son (Glasgow and London), 1991, pp. 116-178), the disclosure of which is incorporated by reference herein, and, in the context of the present invention, their nature is not a critical feature. Thus, nonionic surfactants can include alcohols, α-diols, and alkylphenols. Additionally, copolymers of ethylene oxide, copolymers of propylene oxide, condensates of ethylene oxide with fatty alcohols, condensates of propylene oxide with fatty alcohols, polyethoxylated fatty amides, such as those comprising from 2 to 30 mol of ethylene oxide, polyglycerolated fatty amides on average comprising 1 to 5 glycerol groups, such as from 1.5 to 4, oxyethylenated fatty acid esters of sorbitan comprising from 2 to 30 mol of ethylene oxide, fatty acid esters of sucrose, fatty acid esters of polyethylene glycol, alkylpolyglycosides, N-alkylglucamine derivatives, amine oxides, such as the oxides of (C₁₀-C₁₄) alkylamines, and N-acylaminopropylmorpholine oxides can also be used. It will be noted that the alkylpolyglycosides are nonionic surfactants that can be suitable in the context of the present invention.

(iii) Amphoteric and Zwitterionic Surfactant(s)

Representative amphoteric and zwitterionic surfactants, whose nature is not a critical feature in the context of the present invention, can be chosen from aliphatic secondary and tertiary amine derivatives in which the aliphatic radical is chosen from linear and branched chain radicals comprising 8 to 18 carbon atoms and comprising at least one water-soluble anionic group (chosen for example from carboxylate, sulfonate, sulfate, phosphate and phosphonate); mention may also be made of (C₈-C₂₀)alkylbetaines, sulfobetaines, (C₈-C₂₀)alkylamido(C₁-C₆)alkylbetaines and (C₈-C₂₀)alkylamido(C₁-C₆)alkylsulfobetaines.

Representative amine derivatives include the products sold under the name MIRANOL, as described in U.S. Pat. Nos. 2,528,378 and 2,781,354, the disclosures of which are incorporated by reference herein, and classified in the CTFA dictionary, 3^{rd} edition, 1982, under the names Amphocarboxyglycinates and Amphocarboxypropionates, having the respective structures:

R₂—CONHCH₂CH₂—N⁺(R₃)(R₄)(CH₂COO—) (2)

in which:

R₂ is chosen from alkyl groups derived from an acid R₂—COOH present in hydrolysed coconut oil, and heptyl, nonyl and undecyl radicals,

R₃ is a β-hydroxyethyl group and

R₄ is a carboxymethyl group; and

R₅—CONHCH₂CH₂—N(B)(C) (3)

in which:

(B) is —CH₂CH₂OX′, wherein X′ is an entity chosen from a —CH₂CH₂—COOH group and a hydrogen atom,

(C) is —(CH₂)_{z}—Y′, wherein z=1 or 2, and wherein Y′ is an entity chosen from —COOH and —CH₂—CHOH—SO₃H groups,

R₅ is chosen from alkyl groups, such as (a) alkyl groups of an acid R₅—COOH present in oils chosen from coconut oil and hydrolysed linseed oil, (b) alkyl groups, such as C₇, C₉, C₁₁ and C₁₃ alkyl groups, and (c) C₁₇ alkyl groups and the iso forms, and unsaturated C₁₇ groups.

Such representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, mention may be made of the cocoamphodiacetate sold under the trade name MIRANOL C2M Concentrate by the company RHODIA CHIMIE.

(iv) Cationic Surfactants

Representative cationic surfactants include salts of optionally polyoxyalkylenated primary, secondary and tertiary fatty amines; quaternary ammonium salts such as tetraalkylammonium, alkylamidoalkyltrialkylammonium, trialkylbenzylammonium, and trialkylhydroxyalkylammonium, alkylpyridinium chlorides, alkylpyridinium bromides, imidazoline derivatives; and amine oxides of cationic nature.

The surfactants may be present in the composition according to the invention generally in an amount approximately ranging from 0.01% to 40%, such as approximately from 0.5% to 30% of the total weight of the composition.

The composition according to the present invention may eventually comprise at least one other agent for adjustment of rheology, such as agents chosen from cellulose thickeners (for example, hydroxyethylcellulose, hydroxypropylcellulose, and carboxymethylcellulose), guar gum and its derivatives (for example, hydroxypropylguar), gums of microbial origin (for example, xanthan gum and scleroglucan gum), and synthetic thickeners (for example, crosslinked homopolymers of acrylic acid and crosslinked homopolymers of acrylamidopropanesulfonic acid, and ionic and nonionic associative polymers such as the polymers commercialized under the trade name PEMULEN TR1 and TR2 by the company GOODRICH, SALCARE SC90 by the company ALLIED COLLOIDS and ACULYN 22, 28, 33, 44 and 46 by the company ROHM and HAAS, and ELFACOS T210 and T212 by the company AKZO).

Generally, these thickeners may be present in an amount approximately ranging from 0.01% to 10% by weight of the total weight of the composition.

The medium of the composition appropriate for dyeing can be an aqueous medium, optionally comprising at least one cosmetically acceptable organic solvent.

Representative organic solvents may be chosen from alcohols, such as ethyl alcohol, isopropyl alcohol, benzyl alcohol, and phenylethyl alcohol. The organic solvents may also be chosen from glycols (for example, ethyleneglycol, propyleneglycol, butyleneglycol, dipropyleneglycol, and diethyleneglycol) and ethers of glycols (for example, monomethyl, monoethyl and monobutyl ethers of ethyleneglycol and for example monomethyl ether of propyleneglycol and alkyl ethers of diethyleneglycol glycol, such as, for example, monoethylether and monobutylether of diethyleneglycol).

The organic solvents are generally present in an amount ranging from approximately 0.5% to 20%, such as from approximately 2% to 10% by weight of the total weight of the composition.

Said composition (A) may further comprise an effective quantity of other agents. For example, agents that are already known for oxidation coloration, such as various ordinary adjuvants including sequesterizers such as EDTA and etidronic acid, UV filters, waxes, volatile and nonvolatile, cyclic and non-cyclic, linear and branched, organomodified (especially by amine groups) silicones, preservatives, ceramides, pseudoceramides, vegetable, mineral and synthetic oils, vitamins and provitamins such as panthenol, and opacifiers, may be included.

The composition can also comprise at least one agent chosen from reducing agents and antioxidants. Representative agents may include sodium sulfite, thioglycolic acid, thiolactic acid, sodium bisulfite, dehydroascorbic acid, hydroquinone, 2-methylhydroquinone, tert-butylhydroquinone and homogentisic acid. Such agents may be present in the an amount ranging generally from approximately 0.05% to 1.5% by weight, relative to the total weight of the composition.

One skilled in the art should take care to select said optionally complementary compounds, such that the advantageous properties intrinsically associated with the dye composition according to the invention are not, or are not substantially, adversely affected by the additions envisaged.

In the composition ready for use in the composition (B), the oxidizing agent can be chosen, for example, from urea peroxide, alkali metal bromates and ferricyanides, and persalts such as perborates and persulfates. More specifically, hydrogen peroxide may be used. This oxidizing agent is advantageously constituted by an oxygenated aqueous solution of which the titre may range approximately from 1 to 40 in volume, such as from approximately 5 to 40.

As an oxidizing agent, at least one oxidation-reduction enzyme such as laccases, peroxydases and 2-electron oxydoreductases (such as uricase), if necessary in the presence of their respective donner or cofacter, may also be used.

The pH of the composition ready for use on the keratin fibers [composition resulting from mixing the dye composition (A) and the oxidizing composition (B)], generally ranges from 4 to 11, such as from 6 to 10, and may be adjusted to the desired value by means of at least one agent chosen from acidifying and basifying agents well-known in the art of dyeing keratin fibers.

Representative basifying agents include aqueous ammonia, alkaline carbonates, alkanolamines such as mono-, di- and triethanolamines and derivatives thereof, hydroxyalkylamines and oxyethylenated and oxypropylenated ethylenediamines, sodium and potassium hydroxide and compounds of formula (XIV):

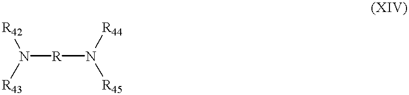

wherein:

R is a propylene residue optionally substituted with a group chosen from hydroxyl and C₁-C₄ alkyl groups;

R₄₂, R₄₃, R₄₄ and R₄₅, which may be identical or different, are each chosen from hydrogen, C₁-C₄ alkyl groups and C₁-C₄ hydroxyalkyl groups.

Representative acidifying agents include, classically, by way of example, organic and inorganic acids such as hydrochloric acid, orthophosphoric acid, and carboxylic acids such as tartaric acid, citric acid, lactic acid and sulfonic acids.

Examples illustrating the invention are found below, in order to describe, without limitation, the invention.

### EXAMPLE 1

A dye composition according to the invention was prepared as follows:

Composition A′

| | |
|---|---|
| Oxyethylenated fatty alcohols | 21 |
| Lauric acid | 3 |
| Cetylstearyl alcohol | 11.5 |
| Polyacrylic acid | 0.4 |
| Silica | 1.2 |
| Opacifying agent | 2 |
| Propylene glycol | 10 |
| Cationic polymer of formula (W) in | 5 |
| a 60% aqueous solution | |
| Merquat 280 | 3.7 |
| Sequestering agent | qs |
| Reducing agent | qs |
| Ammonia (20% NH₃) | 11 |
| Oxidation dye | qs |
| Water qsp | 100 |

Composition A″

| | | |
|---|---|---|
| Ceteareth 60 myristyl glycol | 6.0 | g |
| (ELFACOS GT 282 S of company AKZO) | | |
| Diisopropyl adipate | 50 | g |
| Benzoate of C₁₂-C₁₅ alcohol | 10 | g |
| Preservatives | qs | |
| Water qsp | 100 | |

At the moment of use, 10 g of composition A′ was mixed with 1 g of composition A″ and 15 g of oxygenated water solution at 20 volumes.

A thick and stable composition was obtained. Then, the composition obtained was applied to locks of permed hair containing 90% white hairs. After pausing 30 minutes, the locks were rinsed, then washed with shampoo, rinsed again and then dried. The hair was dyed to a natural brown color.

### EXAMPLE 2

A dye composition according to the invention was prepared as follows:

| | |
|---|---|
| ELFACOS GT 282 S | 1 |
| Oxyethylenated fatty alcohols | 21 |
| Lauric acid | 3 |
| Cetylstearyl alcohol | 11.5 |
| Polyacrylic acid | 0.4 |
| Silica | 1.2 |
| Opacifying agent | 2 |
| Propylene glycol | 10 |
| Cationic polymer of formula (W) in | 5 |
| a 60% aqueous solution | |
| Merquat 280 | 3.7 |
| Sequestrating agent | qs |
| Reducing agent | qs |
| Ammonia (20% NH₃) | 11 |
| Oxidation dye | qs |
| Water qsp | 100 |

At the moment of use, this composition was mixed with oxygenated water solution at 20 volumes. A thick and stable composition was obtained. Then the composition obtained was applied to locks of permed hair containing 90% white hairs. After 30 minutes of pause, the locks of hair were rinsed, then washed with shampoo, rinsed again and then dried.

The hair was dyed to a natural brown color.

What is claimed is:

1. A composition for dyeing keratin fibers comprising (1) at least one colorant chosen from oxidation dyes and direct dyes, and (2) at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100.

2. A composition according to claim 1, wherein said keratin fibers are chosen from human keratin fibers.

3. A composition according to claim 2, wherein human keratin fibers are chosen from human hair.

4. A composition according to claim 1, wherein said R of said at least one nonionic compound is chosen from C₁₆-C₁₈ alkyl groups, and wherein said R′ is chosen from C₁₆-C₁₈ alkyl groups optionally substituted with a hydroxyl radical, and wherein n is an integer equal to or larger than 20.

5. A composition according to claim 1, wherein said R of said at least one nonionic compound is chosen from C₁₆-C₁₈ groups, and wherein said R′ is chosen from C₁₄OH groups.

6. A composition according to claim 5, wherein said n is equal to 60.

7. A composition according to claim 5, wherein said R and R′ of said at least one nonionic compound are chosen from C₁₆-C₁₈ alkyl groups, and said n is equal to 4.

8. A composition according to claim 1, wherein said at least one nonionic compound is present in an amount ranging approximately from 0.05% to 10% by weight relative to the total weight of the composition.

9. A composition according to claim 8, wherein said at least one nonionic compound is present in an amount ranging approximately from 0.1% to 5% by weight relative to the total weight of the composition.

10. A composition according to claim 1, wherein said at least one colorant is chosen from oxidation dyes.

11. A composition according to claim 10, wherein said oxidation dyes are chosen from oxidation bases, oxidation couplers, and oxidation bases and oxidation couplers.

12. A composition according to claim 10, wherein said oxidation dyes are chosen from oxidation bases.

13. A composition according to claim 11, wherein said oxidation bases are chosen from paraphenylenediamines, double bases, ortho- and para-aminophenols and heterocyclic bases and the acid addition salts of the foregoing.

14. A composition according to claim 13, wherein said para-phenylenediamines are chosen from compounds of formula (I), and their acid addition salts: 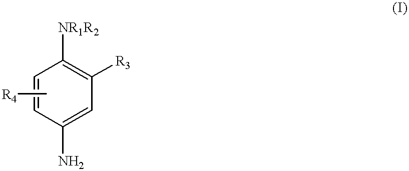
wherein:
R₁ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, phenyl groups, 4′-aminophenyl groups, and C₁-C₄ alkyl groups substituted with at least one group chosen from nitrogen-containing groups;
R₂ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, and C₁-C₄ alkyl groups substituted with a nitrogen-containing group;
R₁ and R₂ may also form, together with the nitrogen atom to which they are bonded, a 5- or 6-membered nitrogen-containing heterocycle ring, optionally substituted with at least one group chosen from alkyl groups, hydroxyl groups and ureido groups;
R₃ is chosen from hydrogen, halogens, such as chlorine, bromine, iodine and fluorine, C₁-C₄ alkyl groups, hydroxy(C₁-C₄ alkyl) groups, hydroxy(C₁-C₄ alkoxy) groups, acetylamino(C₁-C₄ alkoxy) groups, mesylamino(C₁-C₄ alkoxy) groups, and carbamoylamino(C₁-C₄ alkoxy) groups;
R₄ is chosen from hydrogen, halogens, and C₁-C₄ alkyl groups.

15. A composition according to claim 13, wherein said para-phenylenediamines are chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-3-methylaniline, 4-amino-3-chloro-N,N-bis(β-hydroxyethyl)-aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, and their acid addition salts.

16. A composition according to claim 13, wherein said para-phenylenediamines are chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and their acid addition salts.

17. A composition according to claim 13, wherein said double bases are chosen from compounds comprising at least two aromatic rings substituted with at least one group chosen from amino and hydroxyl groups.

18. A composition according to claim 13, wherein said double bases are chosen from compounds of formula (II), and their acid addition salts: 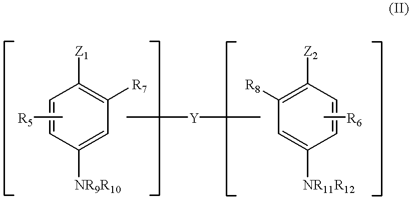
wherein:
Z₁ and Z₂, which are identical or different, are each chosen from hydroxyl groups, and —NH₂ groups, optionally substituted with a group chosen from C₁-C₄ alkyl groups, and linkers Y;
linker Y is chosen from linear and branched, divalent alkylene groups comprising from 1 to 14 carbon atoms, optionally interrupted by, or optionally terminating with, at least one entity chosen from nitrogen-containing groups and heteroatoms such as oxygen, sulfur, and nitrogen, and optionally substituted with at least one group chosen from hydroxyl groups, and C₁-C₆ alkoxy groups;
R₅ and R₆, which are identical or different, are each chosen from hydrogen, halogens, C₁-C₄ alkyl groups, monohydroxy(C₁-C₄ alkyl) groups, polyhydroxy(C₂-C₄ alkyl) groups, amino(C₁-C₄ alkyl) groups, and linkers Y;
R₇, R₈, R₉, R₁₀, R₁₁ and R₁₂, which are identical or different, are each chosen from hydrogen, linkers Y, and C₁-C₄ alkyl groups;
provided that said compounds of formula (II) comprise only one linker Y per molecule.

19. A composition according to claim 13, wherein said double bases are chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their acid addition salts.

20. A composition according to claim 13, wherein said double bases are chosen from N,N′-bis(β-hydroxyethyl)-N,N′-bis(4′-aminophenyl)-1,3-diaminopropanol, 1,8-bis(2,5-diaminophenoxy)-3,5-dioxaoctane, and their acid addition salts.

21. A composition according to claim 13, wherein said para-aminophenols are chosen from compounds of formula (III), and their acid addition salts: 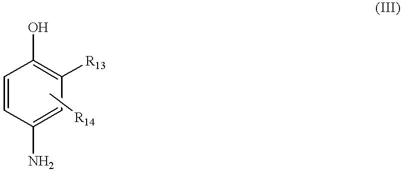
wherein:
R₁₃ is chosen from hydrogen, halogens, such as fluorine, C₁-C₄ alkyl groups, hydroxy(C₁-C₄ alkyl) groups, (C₁-C₄)alkoxy(C₁-C₄)alkyl groups, amino(C₁-C₄)alkylamino(C₁-C₄ alkyl) groups, and hydroxy(C₁-C₄)alkylamino(C₁-C₄ alkyl) groups;
R₁₄ is chosen from hydrogen, halogens, C₁-C₄ alkyl groups, monohydroxy(C₁-C₄ alkyl) groups, polyhydroxy(C₂-C₄ alkyl) groups, amino(C₁-C₄ alkyl) groups, cyano(C₁-C₄ alkyl) groups, and (C₁-C₄)alkoxy(C₁-C₄)alkyl groups;
it being understood that at least one of R₁₃ and R₁₄ is chosen from hydrogen.

22. A composition according to claim 13, wherein said para-aminophenols are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and their acid addition salts.

23. A composition according to claim 13, wherein said ortho-aminophenols are chosen from 2-aminophenol, 2-amino-1-hydroxy-5-methylbenzene, 2-amino-1-hydroxy-6-methylbenzene, 5-acetamido-2-aminophenol, and their acid addition salts.

24. A composition according to claim 13, wherein said heterocyclic bases are chosen from pyridine derivatives, pyrimidine derivatives, pyrazole derivatives, pyrazolo-pyrimidine derivatives, and their acid addition salts.

25. A composition according to claim 24, wherein said pyridine derivatives are chosen from 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their acid addition salts.

26. A composition according to claim 24, wherein said pyrimidine derivatives are chosen from 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and their acid addition salts.

27. A composition according to claim 24, wherein said pyrazolo-pyrimidine derivatives are chosen from pyrazolo[1,5-a]pyrimidines.

28. A composition according to claim 27, wherein said pyrazolo[1,5-a]pyrimidines are chosen from:
pyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
2-(7-aminopyrazolo[1,5-a]pyrimidin-3-ylamino)ethanol;
2-[(3-aminopyrazolo[1,5-a]pyrimidin-7-yl)-(2-hydroxyethyl)amino]ethanol;
2-[(7-aminopyrazolo[1,5-a]pyrimidin-3-yl)-(2-hydroxyethyl)amino]ethanol;
5,6-dimethylpyrazolo[1,5-a]pyrimidine-3,7-diamine;
2,6-dimethylpyrazolo[1,5-a]pyrimidine-3,7-diamine;
2,5,N7,N7-tetramethylpyrazolo[1,5-a]pyrimidine-3,7-diamine;
their acid addition salts, and their tautomeric forms when a tautomeric equilibrium exists.

29. A composition according to claim 24, wherein said pyrazole derivatives are chosen from 4,5-diamino-1-methylpyrazole, 4,5-diamino-1-hydroxyethylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their acid addition salts.

30. A composition according to claim 11, wherein said oxidation bases are present in an amount ranging approximately from 0.0005% to 12% by weight, relative to the total weight of said at least one colorant.

31. A composition according to claim 11, wherein said couplers are chosen from meta-phenylenediamines, meta-aminophenols, meta-diphenols, mono- and poly-hydroxylated derivatives of naphthalene, sesamol and its derivatives and heterocyclic couplers.

32. A composition according to claim 31, wherein said heterocyclic couplers are chosen from indole couplers, indoline couplers, and pyridine couplers, and their acid addition salts.

33. A composition according to claim 11, wherein said couplers are chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, β-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and their acid addition salts.

34. A composition according to claim 11, wherein said couplers are present in an amount ranging approximately from 0.0001% to 10% by weight relative to the total weight of said at least one colorant.

35. A composition according to claim 34, wherein said couplers are present in an amount ranging approximately from 0.005% to 5% by weight relative to the total weight of said at least one colorant.

36. A composition according to claim 13, wherein said acid addition salts are chosen from hydrochlorides, hydrobromides, sulfates, tartrates, lactates and acetates.

37. A composition according to claim 1, wherein said at least one colorant is chosen from direct dyes.

38. A composition according to claim 37, wherein said direct dyes are chosen from nitrobenzene, anthraquinone, azo, neutral, cationic and anionic dyes, naphthoquinone dyes, triarylmethane dyes, xanthene dyes and Arianors.

39. A composition according to claim 37, wherein said direct dyes are present in an amount ranging approximately from 0.01% to 20% by weight, relative to the total weight of the composition.

40. A composition according to claim 1 further comprising at least one polymer chosen from cationic and amphoteric polymers.

41. A composition according to claim 40, wherein said at least one polymer is present in an amount ranging approximately from 0.01% to 10% by weight, relative to the total weight of the composition.

42. A composition according to claim 41, wherein said at least one polymer is present in an amount ranging approximately from 0.05% to 5% by weight, relative to the total weight of the composition.

43. A composition according to claim 42, wherein said at least one polymer is present in an amount ranging approximately from 0.1% to 3% by weight, relative to the total weight of the composition.

44. A composition according to claim 40, wherein said cationic polymers comprise at least one repeating unit chosen from repeating units of formula (W) and (U):

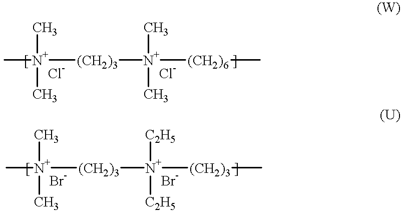

45. A composition according to claim 1 further comprising at least one surfactant.

46. A composition according to claim 45, wherein said at least one surfactant is chosen from anionic, amphoteric, nonionic, zwitterionic and cationic surfactants.

47. A composition according to claim 45, wherein said at least one surfactant is present in an amount ranging approximately from 0.01% to 40% by weight, relative to the total weight of the composition.

48. A composition according to claim 47, wherein said at least one surfactant is present in an amount ranging approximately from 0.5% to 30% by weight, relative to the total weight of the composition.

49. A composition according to claim 1 further comprising at least one thickening agent.

50. A composition according to claim 49, wherein said at least one thickening agent is chosen from cellulose thickeners, guar gum and its derivatives, gums of microbial origin, and synthetic thickeners.

51. A composition according to claim 49, wherein said at least one thickening agent is present in an amount ranging approximately from 0.01% to 10% by weight relative to the total weight of the composition.

52. A composition according to claim 1, further including a medium appropriate for dyeing, wherein said medium appropriate for dyeing is an aqueous medium optionally comprising at least one cosmetically acceptable organic solvent.

53. A composition according to claim 52, wherein said at least one cosmetically acceptable organic solvent is present in an amount ranging approximately from 0.5% to 20%, by weight relative to the total weight of the composition.

54. A composition according to claim 53, wherein said at least one cosmetically acceptable organic solvent is present in an amount ranging approximately from 2% to 10%, by weight relative to the total weight of the composition.

55. A composition according to claim 52, wherein said at least one cosmetically acceptable organic solvent is chosen from alcohols, glycols, ethers of glycols, and alkyl ethers of diethyleneglycol glycol.

56. A composition according to claim 1 further comprising at least one agent chosen from adjuvants, reducing agents and antioxidants.

57. A composition according to claim 56, wherein said at least one agent is present in an amount ranging approximately from 0.05% to 1.5% by weight, relative to the total weight of the composition.

58. A composition according to claim 11, wherein said oxidation dyes comprise at least one oxidation base and at least one coupler.

59. A composition ready to use for dyeing keratin fibers, wherein said composition comprises:
at least one composition (A) comprising, in a medium appropriate for dyeing, at least one colorant chosen from oxidation dyes and direct dyes, and
at least one composition (B) comprising at least one oxidizing agent, and
at least one nonionic compound of the general formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100, wherein said at least one nonionic compound is present in said composition (A), in said composition (B), or in said compositions (A) and (B).

60. A composition according to claim 59, wherein said at least one oxidizing agent of said composition (B) is chosen from hydrogen peroxide, urea peroxide, alkali metal bromates, alkali metal ferricyanides, organic peroxides, persalts and oxidation-reduction enzymes.

61. A composition according to claim 60, wherein said at least one oxidizing agent is present in an oxygenated water solution comprising a titre ranging from 1 to 40 in volume.

62. A composition according to claim 61, wherein said at least one oxidizing agent is present in an oxygenated water solution comprising a titre ranging from 5 to 40 in volume.

63. A composition according to claim 59, wherein said keratin fibers are chosen from human keratin fibers.

64. A composition according to claim 63, wherein said human keratin fibers are chosen from human hair.

65. A composition according to claim 59, said R of said at least one nonionic compound is chosen from C₁₆-C₁₈ alkyl groups, and wherein said R′ is chosen from C₁₆-C₁₈ alkyl groups optionally substituted with a hydroxyl radical, and wherein n is an integer equal to or larger than 20.

66. A composition according to claim 59, said R of said at least one nonionic compound is chosen from C₁₆-C₁₈ groups, and wherein said R′ is chosen from C₁₄OH groups.

67. A composition according to claim 66, wherein said n is equal to 60.

68. A composition according to claim 59, wherein said R and R′ of said at least one nonionic compound are chosen from C₁₆-C₁₈ alkyl groups, and said n is equal to 4.

69. A composition according to claim 59, wherein said at least one nonionic compound is present in an amount ranging approximately from 0.05% to 10% by weight relative to the total weight of the composition.

70. A composition according to claim 69, wherein said at least one nonionic compound is present in an amount ranging approximately from 0.1% to 5% by weight relative to the total weight of the composition.

71. A composition according to claim 59, wherein said at least one colorant is chosen from oxidation dyes.

72. A composition according to claim 71, wherein said oxidation dyes are chosen from oxidation bases, oxidation couplers, and oxidation bases and oxidation couplers.

73. A composition according to claim 72, wherein said oxidation dyes are chosen from oxidation bases.

74. A composition according to claim 72, wherein said oxidation bases are chosen from paraphenylenediamines, double bases, ortho- and para-aminophenols and heterocyclic bases and the acid addition salts of the foregoing.

75. A composition according to claim 74, wherein said para-phenylenediamines are chosen from compounds of formula (I), and their acid addition salts: 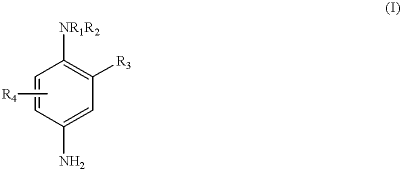
wherein:
R₁ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, phenyl groups, 4′-aminophenyl groups, and C₁-C₄ alkyl groups substituted with at least one group chosen from nitrogen-containing groups,
R₂ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, and C₁-C₄ alkyl groups substituted with a nitrogen-containing group;
R₁ and R₂ may also form, together with the nitrogen atom to which they are bonded, a 5- or 6-membered nitrogen-containing heterocycle ring, optionally substituted with at least one group chosen from alkyl groups, hydroxyl groups and ureido groups;
R₃ is chosen from hydrogen, halogens, such as chlorine, bromine, iodine and fluorine, C₁-C₄ alkyl groups, hydroxy(C₁-C₄ alkyl) groups, hydroxy(C₁-C₄ alkoxy) groups, acetylamino(C₁-C₄ alkoxy) groups, mesylamino(C₁-C₄ alkoxy) groups, and carbamoylamino(C₁-C₄ alkoxy) groups;
R₄ is chosen from hydrogen, halogens, and C₁-C₄ alkyl groups.

76. A composition according to claim 74, wherein said para-phenylenediamines are chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-3-methylaniline, 4-amino-3-chloro-N,N-bis(β-hydroxyethyl)-aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, and their acid addition salts.

77. A composition according to claim 74, wherein said para-phenylenediamines are chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, and their acid addition salts.

78. A composition according to claim 74, wherein said double bases are chosen from compounds comprising at least two aromatic rings substituted with at least one group chosen from amino and hydroxyl groups.

79. A composition according to claim 74, wherein said double bases are chosen from compounds of formula (II), and their acid addition salts: 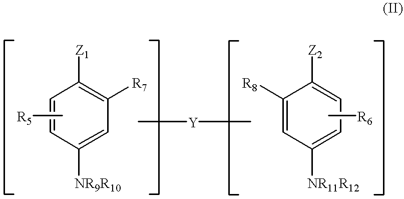
wherein:
Z₁ and Z₂, which are identical or different, are each chosen from hydroxyl groups, and —NH₂ groups, optionally substituted with a group chosen from C₁-C₄ alkyl groups, and linkers Y;
linker Y is chosen from linear and branched, divalent alkylene groups comprising from 1 to 14 carbon atoms, optionally interrupted by, or optionally terminating with, at least one entity chosen from nitrogen-containing groups and heteroatoms such as oxygen, sulfur, and nitrogen, and optionally substituted with at least one group chosen from hydroxyl groups, and C₁-C₆ alkoxy groups;
R₅ and R₆, which are identical or different, are each chosen from hydrogen, halogens, C₁-C₄ alkyl groups, monohydroxy(C₁-C₄ alkyl) groups, polyhydroxy(C₂-C₄ alkyl) groups, amino(C₁-C₄ alkyl) groups, and linkers Y;
R₇, R₈, R₉, R₁₀, R₁₁ and R₁₂, which are identical or different, are each chosen from hydrogen, linkers Y, and C₁-C₄ alkyl groups;
provided that said compounds of formula (II) comprise only one linker Y per molecule.

80. A composition according to claim 74, wherein said double bases are chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their acid addition salts.

81. A composition according to claim 74, wherein said double bases are chosen from N,N′-bis(β-hydroxyethyl)-N,N′-bis(4′-aminophenyl)-1,3-diaminopropanol, 1,8-bis(2,5-diaminophenoxy)-3,5-dioxaoctane, and their acid addition salts.

82. A composition according to claim 74, wherein said para-aminophenols are chosen from compounds of formula (III), and their acid addition salts: 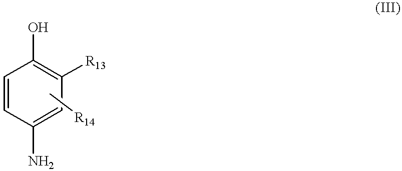
wherein:
R₁₃ is chosen from hydrogen, halogens, such as fluorine, C₁-C₄ alkyl groups, hydroxy(C₁-C₄ alkyl) groups, (C₁-C₄)alkoxy(C₁-C₄)alkyl groups, amino(C₁-C₄)alkylamino(C₁-C₄ alkyl) groups, and hydroxy(C₁-C₄)alkylamino(C₁-C₄ alkyl) groups;
R₁₄ is chosen from hydrogen, halogens, C₁-C₄ alkyl groups, monohydroxy(C₁-C₄ alkyl) groups, polyhydroxy(C₂-C₄ alkyl) groups, amino(C₁-C₄ alkyl) groups, cyano(C₁-C₄ alkyl) groups, and (C₁-C₄)alkoxy(C₁-C₄)alkyl groups;
it being understood that at least one of R₁₃ and R₁₄ is chosen from hydrogen.

83. A composition according to claim 74, wherein said para-aminophenols are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and their acid addition salts.

84. A composition according to claim 74, wherein said ortho-aminophenols are chosen from 2-aminophenol, 2-amino-1-hydroxy-5-methylbenzene, 2-amino-1-hydroxy-6-methylbenzene, 5-acetamido-2-aminophenol, and their acid addition salts.

85. A composition according to claim 74, wherein said heterocyclic bases are chosen from pyridine derivatives, pyrimidine derivatives, pyrazole derivatives, pyrazolo-pyrimidine derivatives, and their acid addition salts.

86. A composition according to claim 85, wherein said pyridine derivatives are chosen from 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their acid addition salts.

87. A composition according to claim 85, wherein said pyrimidine derivatives are chosen from 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and their acid addition salts.

88. A composition according to claim 85, wherein said pyrazolo-pyrimidine derivatives are chosen from pyrazolo[1,5-a]pyrimidines.

89. A composition according to claim 88, wherein said pyrazolo[1,5-a]pyrimidines are chosen from:
pyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
2-(7-aminopyrazolo[1,5-a]pyrimidin-3-ylamino)ethanol;
2-[(3-aminopyrazolo[1,5-a]pyrimidin-7-yl)-(2-hydroxyethyl)amino]ethanol;
2-[(7-aminopyrazolo[1,5-a]pyrimidin-3-yl)-(2-hydroxyethyl)amino]ethanol;
5,6-dimethylpyrazolo[1,5-a]pyrimidine-3,7-diamine;
2,6-dimethylpyrazolo[1,5-a]pyrimidine-3,7-diamine;
2,5,N7,N7-tetramethylpyrazolo[1,5-a]pyrimidine-3,7-diamine;
their acid addition salts, and their tautomeric forms when a tautomeric equilibrium exists.

90. A composition according to claim 85, wherein said pyrazole derivatives are chosen from 4,5-diamino-1-methylpyrazole, 4,5-diamino-1-hydroxyethylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their acid addition salts.

91. A composition according to claim 72, wherein said oxidation bases are present in an amount ranging approximately from 0.0005% to 12% by weight, relative to the total weight of said at least one colorant.

92. A composition according to claim 72, wherein said couplers are chosen from meta-phenylenediamines, meta-aminophenols, meta-diphenols, mono- and poly-hydroxylated derivatives of naphthalene, sesamol and its derivatives and heterocyclic couplers.

93. A composition according to claim 92, wherein said heterocyclic couplers are chosen from indole couplers, indoline couplers, and pyridine couplers, and their acid addition salts.

94. A composition according to claim 72, wherein said couplers are chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, β-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and their acid addition salts.

95. A composition according to claim 72, wherein said couplers are present in an amount ranging approximately from 0.0001% to 10% by weight relative to the total weight of said at least one colorant.

96. A composition according to claim 95, wherein said couplers are present in an amount ranging approximately from 0.005% to 5% by weight relative to the total weight of said at least one colorant.

97. A composition according to claim 74, wherein said acid addition salts are chosen from hydrochlorides, hydrobromides, sulfates, tartrates, lactates and acetates.

98. A composition according to claim 59, wherein said at least one colorant is chosen from direct dyes.

99. A composition according to claim 98, wherein said direct dyes are chosen from nitrobenzene, anthraquinone, azo, neutral, cationic and anionic dyes, naphthoquinone dyes, triarylmethane dyes, xanthene dyes and Arianors.

100. A composition according to claim 98, wherein said direct dyes are present in an amount ranging approximately from 0.01% to 20% by weight, relative to the total weight of the composition.

101. A composition according to claim 59 further comprising at least one polymer chosen from cationic and amphoteric polymers.

102. A composition according to claim 101, wherein said at least one polymer is present in an amount ranging approximately from 0.01% to 10% by weight, relative to the total weight of the composition.

103. A composition according to claim 102, wherein said at least one polymer is present in an amount ranging approximately from 0.05% to 5% by weight, relative to the total weight of the composition.

104. A composition according to claim 103, wherein said at least one polymer is present in an amount ranging approximately from 0.1% to 3% by weight, relative to the total weight of the composition.

105. A composition according to claim 101, wherein said cationic polymers comprise at least one repeating unit chosen from repeating units of formula (W) and (U):

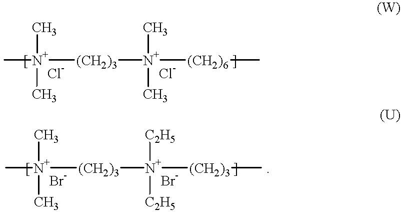

106. A composition according to claim 59 further comprising at least one surfactant.

107. A composition according to claim 106, wherein said at least one surfactant is chosen from anionic, amphoteric, nonionic, zwitterionic and cationic surfactants.

108. A composition according to claim 106, wherein said at least one surfactant is present in an amount ranging approximately from 0.01% to 40% by weight, relative to the total weight of the composition.

109. A composition according to claim 108, wherein said at least one surfactant is present in an amount ranging approximately from 0.5% to 30% by weight, relative to the total weight of the composition.

110. A composition according to claim 59 further comprising at least one thickening agent.

111. A composition according to claim 110, wherein said at least one thickening agent is chosen from cellulose thickeners, guar gum and its derivatives, gums of microbial origin, and synthetic thickeners.

112. A composition according to claim 110, wherein said at least one thickening agent is present in an amount ranging approximately from 0.01% to 10% by weight relative to the total weight of the composition.

113. A composition according to claim 59, wherein said medium appropriate for dyeing is an aqueous medium optionally comprising at least one cosmetically acceptable organic solvent.

114. A composition according to claim 113, wherein said at least one cosmetically acceptable organic solvent is present in an amount ranging approximately from 0.5% to 20%, by weight relative to the total weight of the composition.

115. A composition according to claim 114, wherein said at least one cosmetically acceptable organic solvent is present in an amount ranging approximately from 2% to 10%, by weight relative to the total weight of the composition.

116. A composition according to claim 113, wherein said at least one cosmetically acceptable organic solvent is chosen from alcohols, glycols, ethers of glycols, and alkyl ethers of diethyleneglycol glycol.

117. A composition according to claim 59 further comprising at least one agent chosen from adjuvants, reducing agents and antioxidants.

118. A composition according to claim 117, wherein said at least one agent is present in an amount ranging approximately from 0.05% to 1.5% by weight, relative to the total weight of the composition.

119. A composition according to claim 72, wherein said oxidation dyes comprise at least one oxidation base and at least one coupler.

120. A process for dyeing keratin fibers comprising applying to said keratin fibers at least one composition comprising, in a medium appropriate for dyeing, at least one direct dye and at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100, optionally leaving said composition to act on the fibers for a time ranging from 3 to 60 minutes approximately, rinsing said keratin fibers, optionally washing said keratin fibers, rinsing said keratin fibers again after said optional washing, and drying said keratin fibers.

121. A dyeing process according to claim 120, wherein said keratin fibers are chosen from dry keratin fibers and wet keratin fibers.

122. A dyeing process according to claim 120, wherein said keratin fibers are chosen from human keratin fibers.

123. A dyeing process according to claim 122, wherein said human keratin fibers are human hair.

124. A process for dyeing keratin fibers, comprising applying on said keratin fibers:
at least one composition (A) comprising, in a medium appropriate for dyeing, at least one colorant chosen from oxidation dyes and direct dyes,
at least one oxidizing composition (B), which aids development of said dyeing process at alkaline, neutral or acid pH,
wherein said at least one oxidizing composition (B) is mixed just at the moment of use with said at least one composition (A) or said at least one oxidizing composition (B) is applied sequentially to said at least one composition (A) without intermediate rinsing, and
at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100, wherein said at least one nonionic compound is present in said at least one composition (A), in said at least one composition (B), or in said at least one composition (A) and said at least one composition (B).

125. A process according to claim 124, wherein said oxidation dyes are chosen from bases, couplers, and bases and couplers.

126. A process according to claim 124, wherein said keratin fibers are chosen from human keratin fibers.

127. A process according to claim 126, wherein said human keratin fibers are human hair.

128. A process according to claim 124 comprising extemporaneously preparing at the moment of use a composition comprising at least one composition A and at least one composition B, leaving said composition to act for a time ranging from 1 to 60 minutes approximately, rinsing said keratin fibers, optionally shampooing said keratin fibers, rinsing said keratin fibers after said optional shampooing and drying said keratin fibers.

129. A dyeing process according to claim 128, wherein said keratin fibers are chosen from dry keratin fibers and wet keratin fibers.

130. A dyeing process according to claim 128, wherein said keratin fibers are chosen from human keratin fibers.

131. A dyeing process according to claim 130, wherein said human keratin fibers are human hair.

132. A dyeing process according to claim 128, wherein said time ranges from 10 to 45 minutes approximately.

133. A dyeing process according to claim 124, wherein said oxidation dyes are chosen from bases, couplers, and bases and couplers.

134. A process comprising applying on keratin fibers at least one dyeing composition, leaving said composition on said keratin fibers for a time ranging from 1 to 60 minutes approximately, rinsing said keratin fibers, optionally shampooing said keratin fibers, rinsing said keratin fibers after said optional shampooing, and drying said keratin fibers, wherein said at least one dyeing composition comprises:
at least one composition (A′) comprising at least one colorant chosen from oxidation dyes and direct dyes,
at least one composition (A″) comprising at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100,
at least one oxidizing composition (B)
wherein said at least one dyeing composition comprising said at least one composition (A′), said at least one composition (A″) and said at least one composition (B) is prepared by mixing said at least one compositions (A′), (A″), and (B) at the moment of use.

135. A process according to claim 134, wherein said oxidation dyes are chosen from bases, couplers, and bases and couplers.

136. A process according to claim 134, wherein said keratin fibers are chosen from wet keratin fibers and dry keratin fibers.

137. A process according to claim 134, wherein said time is a time ranging from 10 to 45 minutes approximately.

138. A process according to claim 124, wherein at least one polymer chosen from cationic and amphoteric polymers is present in at least one composition chosen from said at least one composition (A) and said at least one composition (B).

139. A process according to claim 128, wherein at least one polymer chosen from cationic and amphoteric polymers is present in at least one composition chosen from said at least one composition (A) and said at least one composition (B).

140. A process according to claim 134, wherein said at least one dyeing composition comprises at least one polymer chosen from cationic and amphoteric polymers, wherein said at least one polymer is present in at least one composition chosen from said at least one composition (A′), said at least one composition (A″), and said at least one composition (B).

141. A device for dyeing keratin fibers comprising 2 compartments wherein:
a first compartment comprises at least one composition A1 comprising, in a medium appropriate for dyeing, at least one colorant chosen from oxidation dyes and direct dyes,
a second compartment comprises at least one composition B1 comprising, in a medium appropriate for dyeing, at least one oxidizing agent, and wherein
said device comprises, in said at least one composition A1, in said at least one composition B1 or in said at least one composition A1 and in said at least one composition B1, at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and wherein n is an integer ranging from 1 to 100.

142. A device according to claim 141, wherein said oxidation dyes are chosen from bases, couplers, and bases and couplers.

143. A device according to claim 141, wherein said keratin fibers are chosen from human keratin fibers.

144. A device according to claim 143, wherein said human keratin fibers are human hair.

145. A device for dyeing keratin fibers comprising 3 compartments wherein:
a first compartment comprises at least one composition A2 comprising, in a medium appropriate for dyeing, at least one colorant chosen from oxidation dyes and direct dyes,
a second compartment comprises at least one composition B2 comprising, in a medium appropriate for dyeing, at least one oxidizing agent,
a third compartment comprises at least one composition C comprising, in a medium appropriate for dyeing, at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and wherein n is an integer ranging from 1 to 100, and wherein
said at least one composition A2, said at least one composition B2, and said at least one composition A2 and said at least one composition B2 can also further comprise said at least one nonionic compound.

146. A device according to claim 145, wherein said keratin fibers are chosen from human keratin fibers.

147. A process according to claim 146, wherein said human keratin fibers are human hair.

148. A process according to claim 145, wherein said oxidation dyes are chosen from bases, couplers, and bases and couplers.

149. A process for dyeing keratin fibers, comprising applying on said keratin fibers:
at least one composition (A) comprising, in a medium appropriate for dyeing, at least one colorant chosen from oxidation dyes and direct dyes,
at least one oxidizing composition (B), which aids development of said dyeing process at alkaline, neutral or acid pH, and
at least one nonionic compound of the formula R—(OCH₂CH₂)ₙ—OR′, wherein R is chosen from C₁₀-C₃₀ alkyl groups, and wherein R′ is chosen from C₁₀-C₃₀ alkyl groups optionally substituted with a hydroxyl group, and n is an integer ranging from 1 to 100.
